(19) United States
(12) Patent Application Publication
(10) Pub. No.: US 2024/0025857 A1
(43) Pub. Date: Jan. 25, 2024

(54) NEW CATECHOLAMINE PRODRUGS FOR USE IN THE TREATMENT OF PARKINSON'S DISEASE

(71) Applicant: H. Lundbeck A/S, Valby (DK)

(72) Inventors: Morten Jørgensen, (US); Klaus Gjervig Jensen, (US); Jarkko Tapani Rautio, (US); Christoffer Gerner, Bavnhøj Hansen, (US); Jette Bisgaard Boll, (US); Lena Gustavsson, (US); Anja Nørgaard Kjeldsen, (US)

(73) Assignee: H. Lundbeck A/S, Valby (DK)

(21) Appl. No.: 18/036,596

(22) PCT Filed: Nov. 15, 2021
(86) PCT No.: PCT/EP2021/081678

§ 371 Date: May 11, 2023

(30) Foreign Application Priority Data
Nov. 17, 2020 (EP) 20207969.5

(51) Int. Cl. C07D 221/06

(52) U.S. Cl. CPC C07D 221/06

(57) ABSTRACT

The present invention provides sulfamate derivative prodrugs of the dopamine agonist (4aR,10aR)-1-Propyl-1,2,3,4,4a,5,10,10a-octahydro-benzo[g]quinoline-6,7-diol, their use in the treatment of conditions for which treatment with a dopamine agonist is therapeutically beneficial and pharmaceutical compositions comprising compounds of the invention. Compounds according to the invention are of formula (Id), wherein R1 and R2 are each independently selected from H, and substituent (iii) below, wherein * indicates the attachment point to oxygen, wherein R3 is selected from H and COR4, and wherein R4 is selected from H and C1-C6 alkyl, with the proviso that R1 and R2 cannot both be H,15 or a pharmaceutically acceptable salt thereof.

### FIELD OF THE INVENTION

The present invention provides compounds that are sulfamate derivative prodrugs of the dopamine agonist (4aR,10aR)-1-Propyl-1,2,3,4,4a,5,10,10a-octahydro-benzo[g]quinoline-6,7-diol, and their use in the treatment of Parkinson's disease and/or other conditions for which treatment with a dopamine agonist is therapeutically beneficial, for example Restless leg syndrome, Huntington's disease and Alzheimer's disease; and neuropsychiatric diseases and disorders, such as schizophrenia, attention deficit hyperactivity disorder and drug addiction. The present invention further provides pharmaceutical compositions comprising compounds of the invention.

### BACKGROUND OF THE INVENTION

Parkinson's disease (PD) is a common neurodegenerative disorder that becomes increasingly prevalent with age and affects an estimated seven to ten million people worldwide. Parkinson's disease is a multi-faceted disease characterized by both motor and non-motor symptoms. Motor symptoms include resting tremor (shaking), bradykinesia/akinesia (slowness and poverty of movements), muscular rigidity, postural instability and gait dysfunction; whereas non-motor symptoms include neuropsychiatric disorders (e.g. depression, psychotic symptoms, anxiety, apathy, mild-cognitive impairment and dementia) as well as autonomic dysfunctions and sleep disturbances (Poewe et al., Nature Review, (2017) vol 3 article 17013: 1-21).

A key hallmark of Parkinson's disease pathophysiology is the loss of pigmented dopaminergic neurons in the substantia nigra pars compacta that provides dopaminergic innervation to the striatum and other brain areas. Such progressive neurodegeneration leads to the decrease in dopamine striatal levels which ultimately results in a series of changes in the basal ganglia circuitry, ultimately ending up in the occurrence of the four cardinal motor features of Parkinson's disease. The main target of dopamine in the striatum consists of medium spiny GABAergic neurons (MSNs) selectively expressing D1 or D2 receptors pending topographical projections. GABAergic-MSN projecting to the external pallidum, also called striato-pallidal ‘indirect pathway’ express D2 receptors (MSN-2); whereas GABAergic-MSN projecting to the substantia nigra pars reticulata and internal pallidum, also called striato-nigral ‘direct pathway’ express D1 receptors (MSN-1). Depletion of dopamine because of neuronal loss results in an imbalanced activity of the two pathways, resulting in a marked reduction of thalamic and cortical output activities and ultimately motor dysfunctions (Gerfen et al, Science (1990) 250: 1429-32; Delong, (1990) Trends in Neuroscience 13: 281-5; Alexander et Crutcher, (1990) Trends in Neuroscience 13: 266-71; and for review Poewe et al., Nature Review (2017) vol. 3 article 17013: 1-21).

The most effective therapeutic strategies available to patients suffering from Parkinson's disease, and aiming at controlling motor symptoms are primarily indirect and direct dopamine agonists. The classic and gold standard treatment regimen includes chronic oral intake of L-3,4-dihydroxy phenylalanine (L-DOPA) which is decarboxylated in the brain to form dopamine. Other approaches consist in the administration of dopamine receptor agonists such as apomorphine which acts both on the D1 and D2 receptors subtypes, or pramipexole, ropinirole and others which are predominantly directed towards D2 receptors subtypes. Optimal motor relief is obtained with use of both L-DOPA and apomorphine due to their activation of both D1 and D2 receptor subtypes and holistic re-equilibrium of the indirect-direct pathways (i.e. while D2 agonists only reverse the indirect pathway dysfunction).

L-DOPA and apomorphine with the structures depicted below are currently the most efficacious PD drugs in clinical use.

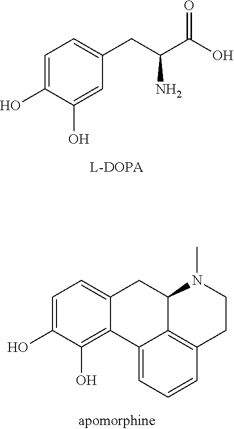 [SMILES: N[C@@H](Cc1ccc(O)c(O)c1)C(=O)O ; CN1CCc2cccc3c2[C@H]1Cc1ccc(O)c(O)c1-3]

L-DOPA is a prodrug of dopamine and remains the most efficacious drug in the treatment of motor Parkinson's disease. However, after several years of treatment (i.e. honeymoon period), complications arise due the inherent progression of the disease (i.e. sustained loss of dopaminergic neurons) as well as poor pharmacokinetic (PK) profile of L-DOPA. Those complications include: 1) dyskinesia which are abnormal involuntary movements occurring during the optimal ‘on-time effect’ of the drug; and 2) off fluctuations, period during which the L-DOPA positive effect wears off and symptoms re-emerge or worsen (Sprenger and Poewe, CNS Drugs (2013), 27: 259-272).

Direct dopamine receptor agonists are able to activate the dopamine auto receptors as well as the postsynaptic dopamine receptors located on the medium spiny neurons MSN-1 and MSN-2. Apomorphine belongs to a class of dopamine agonists with a 1,2-dihydroxybenzene (catechol) moiety. When combined with a phenethylamine motif, catecholamines often possess low or no oral bioavailability as is the case for apomorphine. Apomorphine is used clinically in PD therapy albeit with a non-oral delivery (typically intermittent subcutaneous administration or daytime continuous parenteral infusion via a pump). For apomorphine, animal studies have shown that transdermal delivery or implants may provide possible forms of administration. However, when the delivery of apomorphine from implants was studied in monkeys (Campbell et al et al., Chase Experimental Neurology (2005), 192: 73-78) it was found that in most cases the animals had to be treated with the immunosuppressant dexamethasone to prevent local irritation and other complications following the implantation surgery. Alternative delivery strategies for apomorphine therapy in PD such as inhalation and sublingual formulations have been extensively explored (see e.g. Grosset et al., Acta Neurol Scand. (2013), 128:166-171 and Hauser et al., Movement Disorders (2016), Vol. 32 (9): 1367-1372).

An alternative to the non-oral formulations of the catecholamines involves the use of a prodrug masking the free catechol hydroxyl groups to enable oral administration. However, a known problem associated with the development of prodrugs for clinical use is the difficulties associated with predicting conversion to the parent compound in humans.

Various ester prodrugs of catecholamines have been reported in the literature such as enterically coated N-propyl-noraporphine (NPA) and the mono pivaloyl ester of apomorphine for duodenal delivery (see eg. WO 02/100377), and the D1-like agonist adrogolide, a diacetyl prodrug of A-86929 (Giardina and Williams; CNS Drug Reviews (2001), Vol. 7 (3): 305-316). Adrogolide undergoes extensive hepatic first-pass metabolism in man after oral dosing and, as a result, has a low oral bioavailability (app. 4%). In PD patients, intravenous (IV) adrogolide has antiparkinson efficacy comparable to that of L-DOPA (Giardina and Williams; CNS Drug Reviews (2001), Vol. 7 (3): 305-316).

In addition to the ester prodrugs of catecholamines, an alternative prodrug approach involves the masking of the two catechol hydroxyl groups as the corresponding methylenedioxy (MDO) derivative or di-acetalyl derivative. This prodrug principle has been described for example in Campbell et al., Neuropharmacology (1982); 21(10): 953-961 and in U.S. Pat. No. 4,543,256, WO 2009/026934 and WO 2009/026935.

Yet another suggested approach for a catecholamine prodrug is the formation of an enone derivative as suggested in for example WO 2001/078713 and in Liu et al., Bioorganic Med. Chem. (2008), 16: 3438-3444. For further examples of catecholamine prodrugs see for example Sozio et al., Exp. Opin. Drug Disc. (2012); 7(5): 385-406.

The compound (4aR,10aR)-1-n-Propyl-1,2,3,4,4a,5,10,10a-octahydro-benzo[g]quinoline-6,7-diol depicted as compound (I) below is disclosed in WO 2009/026934. The trans-isomer was disclosed previously in Liu et al., J. Med. Chem. (2006), 49: 1494-1498 and then in Liu et al., Bioorganic Med. Chem. (2008), 16: 3438-3444 including pharmacological data indicating that the compound has a low oral bioavailability in rats. The racemate was disclosed for the first time in Cannon et al., J. Heterocyclic Chem. (1980); 17: 1633-1636.

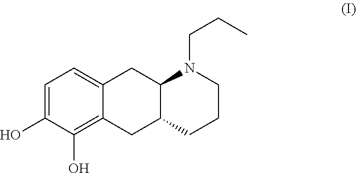 [SMILES: CCCN1CCC[C@@H]2Cc3c(ccc(O)c3O)C[C@H]21]

Compound (I) is a dopamine receptor agonist with mixed D1 and D2 activity. Different prodrug derivatives of compound (I) are known in the art.

Liu et al., J. Med. Chem. (2006), 49: 1494-1498 and Liu et al., Bioorganic Med. Chem. (2008), 16: 3438-3444 disclose the enone derivative of formula (Ia) depicted below which was shown to be converted to the active compound (I) in rats.

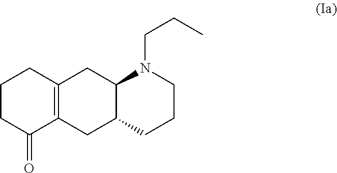 [SMILES: CCCN1CCC[C@@H]2CC3=C(CCCC3=O)C[C@H]21]

WO 2009/026934 and WO 2009/026935 disclose two types of prodrug derivatives of compound (I) including (6aR,10aR)-7-propyl-6,6a,7,8,9,10,10a,11-octahydro-[1,3]dioxolo[4′,5′:5,6]benzo[1,2-g]quinoline, a methylenedioxy (MDO) derivative with the formula (Ib) below:

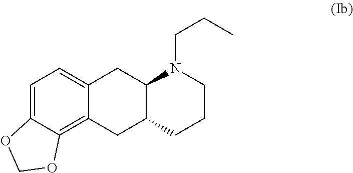 [SMILES: CCCN1CCC[C@@H]2Cc3c(ccc4c3OCO4)C[C@H]21]

The conversion of compound (Ib) to compound (I) in rat and human hepatocytes has been demonstrated in WO 2010/097092. Furthermore, the in vivo pharmacology of the compounds (Ia) and (Ib) as well as the active “parent compound” (I) has been tested in various animal models relevant for Parkinson's Disease (WO 2010/097092). Both compound (I) and compounds (Ia) and (Ib) were found to be effective, indicating that compounds (Ia) and (Ib) are converted in vivo to compound (I). All three compounds were reported to have a duration of action that was longer than observed for L-dopa and apomorphine.

The other prodrug of compound (I) disclosed in WO 2009/026934 and WO 2009/026935 is a conventional ester prodrug of the formula (Ic) shown below:

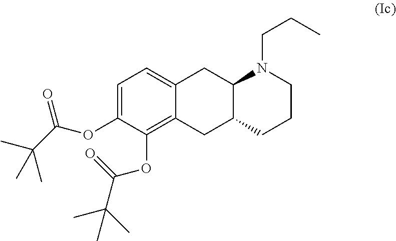 [SMILES: CCCN1CCC[C@@H]2Cc3c(ccc(OC(=O)C(C)(C)C)c3OC(=O)C(C)(C)C)C[C@H]21]

The patent application WO2019101917 discloses further glucuronide conjugates of compound (I), as well as sulfate conjugates of compound (I) of formulas (Id-iia), (Id-iib), and (Id-iiab) below.

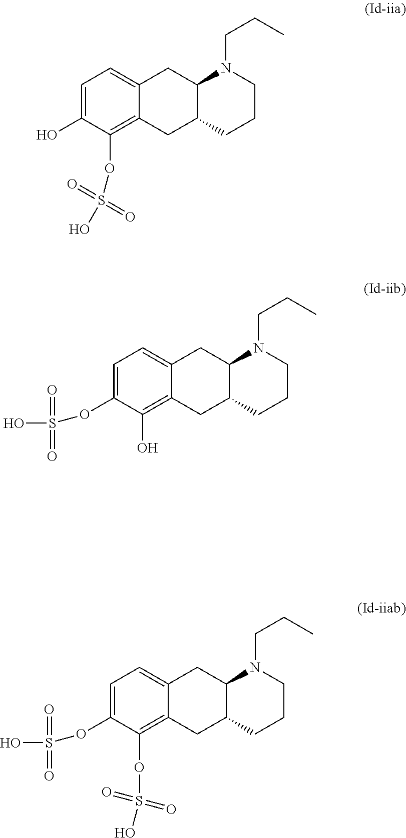 [SMILES: CCCN1CCC[C@@H]2Cc3c(ccc(O)c3OS(=O)(=O)O)C[C@H]21 ; CCCN1CCC[C@@H]2Cc3c(ccc(OS(=O)(=O)O)c3O)C[C@H]21 ; CCCN1CCC[C@@H]2Cc3c(ccc(OS(=O)(=O)O)c3OS(=O)(=O)O)C[C@H]21]

WO2019101917 proposes the use of the disclosed glucuronide and sulfate derivatives of compound (I) as orally active prodrugs of compound (I).

Other conjugates of compound (I) have been disclosed in WO2020234274, WO2020234275, WO2020234276, and WO2020234277.

Despite the long-standing interest in the field, there is evidently still an unmet need for developing efficient, well-tolerated and orally active drugs for the treatment of PD. A prodrug derivative of a mixed D1/D2 agonist having a favorable PK profile which can provide continuous dopaminergic stimulation may fulfil such unmet needs.

Sulfamate derivatives as prodrug principle has previously been suggested, for example for estradiol (Elger et al. J Steroid Biochem Mol Biol 55: 395-403, 1995, and Elger et al. Reprod Fertil Dev 13: 297-305, 2001).

### SUMMARY OF THE INVENTION

The present invention relates to new compounds for treatment of Parkinson's Disease. More particularly, the invention relates to new sulfamate prodrug derivatives of the compound (4aR,10aR)-1-n-Propyl-1,2,3,4,4a,5,10,10a-octahydro-benzo[g]quinoline-6,7-diol (compound (I)). The compounds of the invention have proven particularly useful for oral delivery of compound (I), as demonstrated in rat PK study of Example 2 where it is shown that the tested compounds of the invention are converted into compound (I) in vivo.

Example 2 further demonstrates that the tested compounds (II) and (III) of the invention result in a rat plasma exposure throughout 24 hours that is lower than the corresponding exposure observed for prior art compounds (Ia) and (Ib), while higher than the corresponding exposure for prior art compound (Ic), which was found not to be useful as a prodrug.

Further, the Cmax of compound (I) values observed after dosing both compounds (II) and (III) in rats are lower than what is achievable from dosing prior art compounds (Ia) and (Ib), and the prior art sulfate conjugate compounds (Id-iia), and (Id-iib). Since the peak concentrations of compound (I) which are expected to drive the side effects are lower, higher doses might be administered of the compounds of the invention to potentially achieve higher overall plasma concentrations of compound (I) compared to what is achievable from dosing compounds (Ia) and (Ib). Compound (II) had observed Tmax after about 1 hour, which was faster than the prior art sulfate conjugate compounds (Id-iia), (Id-iib) and (Id-iiab), and at the same time comparable to the prior art compounds (Ia), (Ib) and (Ic).

Example 4 demonstrates that compounds (II) and (III) are converted to compound (I) by incubation with human and rat liver S9 fractions (FIGS. 3 and 4). Further, compounds (II) and (III) of the invention had increased conversion to compound (I) by incubation with human liver S9 compared to prior art sulfate conjugate compounds (Id-iia) and (Id-iib) (FIG. 3).

Further, Example 3 demonstrates that the solubility at pH 6 of compounds of the invention, particularly compound (II) and compound (III), is higher than the solubility of the corresponding prior art sulfate conjugate compounds (Id-iia) and (Id-iib). A high solubility at pH 6.0 and lower pH may be advantageous to facilitate absorption of the compounds after enteral administration.

In a first aspect, the invention provides a compound according to formula (Id) below

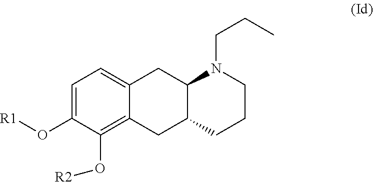

wherein R1 and R2 are each independently selected from H, and substituent (iii) below,

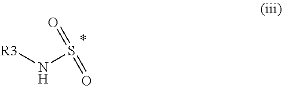

wherein * indicates the attachment point to oxygen,
wherein R3 is selected from H and COR4,
and wherein R4 is selected from H and C1-C6 alkyl,
with the proviso that R1 and R2 cannot both be H,
or a pharmaceutically acceptable salt thereof.

In a preferred embodiment of the invention, the compound according to formula (Id) is a compound wherein R3 is H.

In another preferred embodiment of the invention, the compound according to formula (Id) is selected from the group consisting of (4aR,10aR)-6-hydroxy-1-propyl-1,2,3,4,4a,5,10,10a-octahydrobenzo[g]quinolin-7-yl sulfamate of formula (II), and (4aR,10aR)-7-hydroxy-1-propyl-1,2,3,4,4a,5,10,10a-octahydrobenzo[g]quinolin-6-yl sulfamate of formula (III) below

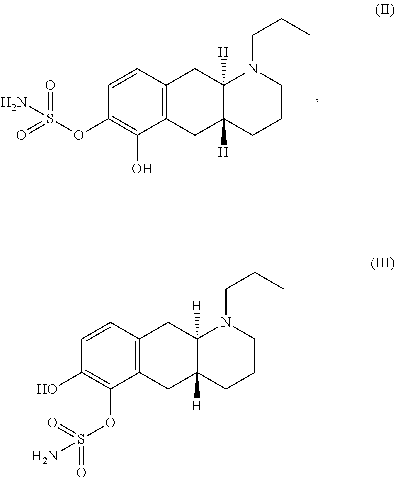 [SMILES: CCCN1CCC[C@@H]2Cc3c(ccc(OS(N)(=O)=O)c3O)C[C@H]21 ; CCCN1CCC[C@@H]2Cc3c(ccc(O)c3OS(N)(=O)=O)C[C@H]21]

and a pharmaceutically acceptable salt thereof.

In other preferred embodiments, the compound according to the invention is in an isolated form substantially free of the compound of formula (I). In other embodiments of the invention, said compound or pharmaceutically acceptable salt thereof is in a solid form.

A further aspect of the invention provides a pharmaceutically acceptable salt of a compound according to formula (Id).

A further aspect of the invention provides a compound of formula (Id) or pharmaceutically acceptable salt thereof for use as a medicament.

A further aspect of the invention provides a pharmaceutical composition comprising a therapeutically effective amount of a compound according to formula (Id) or pharmaceutically acceptable salt thereof, and one or more pharmaceutically acceptable excipients. In a preferred embodiment, said pharmaceutical composition is an oral pharmaceutical composition such as a tablet or a capsule for oral administration.

A further aspect of the invention provides a compound of formula (Id) or pharmaceutically acceptable salt thereof for use in the treatment of a neurodegenerative disease or disorder such as Parkinson's Disease, Huntington's disease, Restless leg syndrome or Alzheimer's disease; or a neuropsychiatric disease or disorder such as schizophrenia, attention deficit hyperactivity disorder or drug addiction. In a preferred embodiment, a compound of formula (Id) or pharmaceutically acceptable salt thereof is for use in treatment of Parkinson's Disease.

A further aspect of the invention provides the use of a compound according to formula (Id) or pharmaceutically acceptable salt thereof, or a pharmaceutical composition as described above in the manufacture of a medicament for the treatment of neurodegenerative disease or disorder such as Parkinson's Disease, Huntington's disease, Restless leg syndrome or Alzheimer's disease; or a neuropsychiatric disease or disorder such as schizophrenia, attention deficit hyperactivity disorder or drug addiction. In a preferred embodiment, the medicament is for the treatment of Parkinson's Disease.

A further aspect of the invention provides a method for the treatment of a neurodegenerative disease or disorder such as Parkinson's Disease, Huntington's disease, Restless leg syndrome or Alzheimer's disease; or a neuropsychiatric disease or disorder such as schizophrenia, attention deficit hyperactivity disorder or drug addiction; which method comprises the administration of a therapeutically effective amount of a compound according to formula (Id) or pharmaceutically acceptable salt thereof, to a patient in need thereof.

### BRIEF DESCRIPTION OF FIGURES

FIG. 1

PK profile in Wistar rats obtained after oral dosing according to Example 2. The profile is based on mean plasma concentrations from 3 subjects per time point for each compound. X-axis: time (hours); Y-axis: plasma concentration of Compound (I) (pg/mL) obtained after dosing of compound (III).

FIG. 2

PK profile in Wistar rats obtained after oral dosing according to Example 2. The profile is based on mean plasma concentrations from 3 subjects per time point for each compound. X-axis: time (hours); Y-axis: plasma concentration of Compound (1) (pg/mL) obtained after dosing of compound (II).

FIG. 3

In vitro conversion of compounds (II), (III), (Id-iia) and (Id-iib) to compound (I) in human liver S9.

X-axis: Time (min); Y-axis: Increase in concentration of compound (I) (pg/mL) from concentration at time=0 min

Filled black circles denote compound (III), open circles denote compound (II), crosses (X) denote compound (Id-iia), and triangles denote compound (Id-iib).

FIG. 4

In vitro conversion of compounds (II), (III), (Id-iia) and (Id-iib) to compound (I) in rat liver S9. X-axis: Time (min); Y-axis: Increase in concentration of compound (I) (pg/mL) from time=0 min. Filled black circles denote compound (III), open circles denote compound (II), crosses (X) denote compound (Id-iia), and triangles denote compound (Id-iib).

### DETAILED DESCRIPTION OF THE INVENTION

### Definitions

Compounds

Reference to compounds encompassed by the invention includes the free substance (e.g. a free base or a zwitterion) of compounds of the invention, pharmaceutically acceptable salts of compounds of the invention, such as acid addition salts or base addition salts, and polymorphic and amorphic forms of compounds of the invention and of pharmaceutically acceptable salts thereof. Furthermore, the compounds of the invention and pharmaceutically acceptable salts thereof may potentially exist in nonsolvate as well as in solvated forms with pharmaceutically acceptable solvents such as water, ethanol and the like.

Both solvated and unsolvated forms are encompassed by the present invention.

Combination Treatment

The terms “combined use”, “in combination with” and “a combination of” and the like as used herein in the context of the method of the invention comprising the combined administration of therapeutically effective amounts of a compound of formula (Id), and another compound, which compound is useful in the treatment a neurodegenerative disease or disorder, is intended to mean the administration of a compound of formula (Id) simultaneously or sequentially, in any order, together with said other compound.

Pharmaceutically Acceptable Salts

The compounds of this invention are generally utilized as the free substance or as a pharmaceutically acceptable salt thereof. When a compound of formula (Id) contains a free base, such salts may be prepared in a conventional manner by treating a solution or suspension of a free base of formula (Id) with a molar equivalent of a pharmaceutically acceptable acid. Representative examples of suitable organic and inorganic acids are described below.

Pharmaceutically acceptable salts in the present context is intended to indicate non-toxic, i.e. physiologically acceptable salts.

The term “pharmaceutically acceptable salts” include pharmaceutically acceptable acid addition salts which are salts formed with inorganic and/or organic acids on the nitrogen atom in the parent molecule. Said acids may be selected from for example hydrochloric acid, hydrobromic acid, phosphoric acid, nitrous acid, sulphuric acid, benzoic acid, citric acid, gluconic acid, lactic acid, maleic acid, succinic acid, tartaric acid, acetic acid, propionic acid, oxalic acid, malonic acid, fumaric acid, glutamic acid, pyroglutamic acid, salicylic acid, gentisic acid, saccharin, and sulfonic acids such as methanesulfonic acid, ethanesulfonic acid, toluenesulfonic acid, naphthalene-2-sulphonic acid, 2-hydroxy ethanesulphonic acid and benzenesulfonic acid.

Additional examples of useful acids and bases to form pharmaceutically acceptable salts can be found e.g. in Stahl and Wermuth (Eds) “Handbook of Pharmaceutical salts. Properties, selection, and use”, Wiley-VCH, 2008.

Prodrug

In the present context, the terms “prodrug” or “prodrug derivative” indicates a compound that, after administration to a living subject, such as a mammal, preferably a human; is converted within the body into a pharmacologically active moiety. The conversion preferably takes place within a mammal, such as in a mouse, rat, dog, minipig, rabbit, monkey and/or human. In the present context a “prodrug of the compound (4aR,10aR)-1-n-Propyl-1,2,3,4,4a,5,10,10a-octahydro-benzo[g]quinoline-6,7-diol” or “a prodrug of the compound of formula (I)” or “a prodrug of compound (I)” is understood to be a compound that, after administration, is converted within the body into the compound (4aR,10aR)-1-n-Propyl-1,2,3,4,4a,5,10,10a-octahydro-benzo[g]quinoline-6,7-diol. Said administration may be by any conventional route of administration of pharmaceutical compositions known in the art, preferably by oral administration.

In the present context, the terms “parent compound” and “parent molecule” indicate the pharmacologically active moiety obtained upon conversion of a corresponding prodrug. For example, the “parent compound” of one of the compounds (Ia), (Ib), (Ic) or any of the compounds of the invention according to formula (Id) (e.g. compounds (II), (III) and (V)) is understood to be the compound of formula (I).

Substituents

In the present context, a given range may interchangeably be indicated with “-” (dash) or “to”, e.g. the term “C1-C6 alkyl” is equivalent to “C1 to C6 alkyl”.

The term “alkyl” refers to a linear (i.e. unbranched) or branched saturated hydrocarbon having from one up to six carbon atoms, inclusive. Examples of such groups include, but are not limited to, methyl, ethyl, 1-propyl, 2-propyl, 1-butyl, 2-butyl, 2-methyl-2-propyl, 2-methyl-1-butyl, n-pentyl, isopentyl and n-hexyl.

### Pharmacokinetic Definitions and Abbreviations

As used herein, a “PK profile” is an abbreviation of “pharmacokinetic profile”. Pharmacokinetic profiles and pharmacokinetic parameters described herein are based on the plasma concentration-time data obtained for the compound of formula (I) after oral dosing of a compound of the invention, using non-compartmental modelling. Abbreviated PK parameters are: Cmax (maximum concentration); tmax (time to Cmax); t½ (half-life); AUC0-24 (area under the curve from time of dosing and until 24 hours after dosing), and “Exposure at 24 h” is the plasma concentration of the compound of formula (I) as measured 24 hours after dosing.

Therapeutically Effective Amount

In the present context, the term “therapeutically effective amount” of a compound of the invention means an amount sufficient to alleviate, arrest, partly arrest, remove or delay the clinical manifestations of a given disease and its complications in a therapeutic intervention comprising the administration of said compound. An amount adequate to accomplish this is defined as “therapeutically effective amount”. Effective amounts for each purpose will depend e.g. on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician.

In the context of the present invention, a “therapeutically effective amount” of a compound of the invention indicates an amount of said compound of the invention that is able to provide an amount of compound (I) that is sufficient to alleviate, arrest, partly arrest, remove or delay the clinical manifestations of a given disease and its complications when said compound of the invention is administered, preferably by the oral route, to a mammal, preferably to a human being.

Treatment and Treating

In the present context, “treatment” or “treating” is intended to indicate the management and care of a patient for the purpose of alleviating, arresting, partly arresting, removing or delaying progress of the clinical manifestation of the disease. The patient to be treated is preferably a mammal, in particular a human being.

Pharmaceutical Formulations and Excipients

In the following, the term, “excipient” or “pharmaceutically acceptable excipient” refers to pharmaceutical excipients including, but not limited to, carriers, fillers, diluents, antiadherents, binders, coatings, colours, disintegrants, flavours, glidants, lubricants, preservatives, sorbents, sweeteners, solvents, vehicles and adjuvants.

Headings and sub-headings are used herein for convenience only and should not be construed as limiting the invention in any way.

The description herein of any aspect or aspect of the invention using terms such as “comprising”, “having,” “including” or “containing” with reference to an element or elements is intended to provide support for a similar aspect or aspect of the invention that “consists of”, “consists essentially of” or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

The use of any and all examples, or exemplary language (including “for instance”, “for example”, “e.g.”, “such as” and “as such”) in the present specification is intended merely to better illuminate the invention and does not pose a limitation on the scope of invention unless otherwise indicated.

It should be understood that the various aspects, embodiments, implementations and features of the invention mentioned herein may be claimed separately, or in any combination.

The present invention includes all modifications and equivalents of the subject-matter recited in the claims appended hereto, as permitted by applicable law.

### Compounds of the Invention

The inventors have identified new sulfamate compounds that may function as prodrugs of (4aR,10aR)-1-Propyl-1,2,3,4,4a,5,10,10a-octahydro-benzo[g]quinoline-6,7-diol [compound (I)] which is a dual D1/D2 agonist (see for example WO 2009/026934).

It was found that oral dosing of representative compounds (II) and (III) of the invention in Wistar rats provides a systemic exposure of compound (I) as measured by the concentration of compound (I) in plasma, suggesting the usefulness of said compounds as orally active prodrugs of compound (I).

For the compounds tested in vivo, the doses were corrected by molecular weight to equal a dose of 300 microgram/kg of compound (Ib) corresponding to 287 microgram/kg of compound (I).

It has previously been observed that oral dosing of compounds (Ia) and (Ib) to Wistar rats results in early and high peak plasma concentrations of compound (I). Such high peak concentrations are in humans likely to be associated with dopaminergic side effects such as for example nausea, vomiting and light headedness. In contrast, for the compound (II) of the invention a slower absorption rate was observed accompanied by a sustained exposure of compound (I) avoiding rapid peak concentrations. Additionally, the plasma exposure of compound (I) after oral administration of compounds (II) and (III) in Wistar rats as shown in Example 2 herein shows a moderate exposure although higher than observed for compound (Ic). However, since the peak concentrations of compound (I) which are expected to drive the side effects are low, higher doses might be administered of the compounds of the invention to potentially achieve higher overall plasma concentrations of compound (I) compared to what is achievable from dosing compounds (Ia) and (Ib). When investigating PK properties of compound (Ic), the inventors found that the plasma concentrations of compound (I) were extremely low, leaving compound (Ic) unsuitable as a prodrug of compound (I) for oral administration and confirming that the oral bioavailability of the compounds of the invention is highly unpredictable.

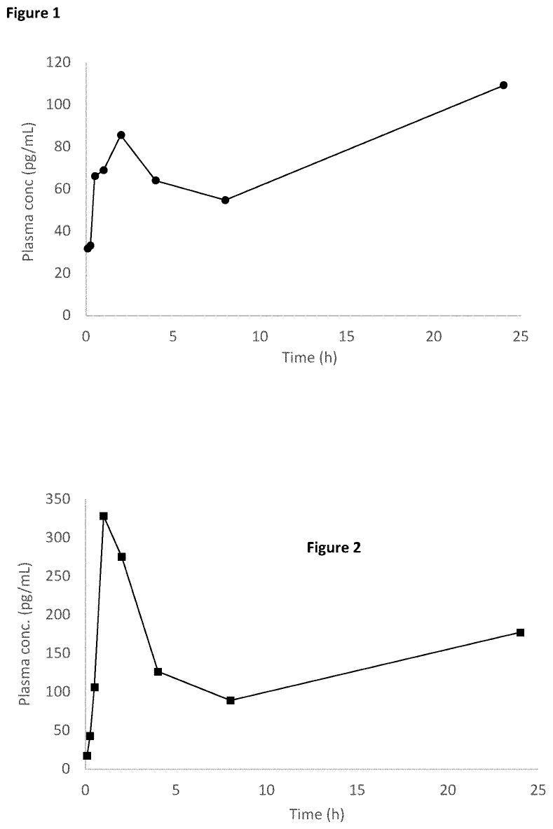

PK parameters for the PK studies of compounds (II) and (III) in Wistar rats are listed in Table 2 and PK profiles are depicted in FIGS. 1 and 2.

Thus, in conclusion, representative compounds of the invention are useful as orally available prodrugs of compound (I) and have been observed in rats to provide a PK profile avoiding the peak Cₘₐₓ observed for the known prodrugs (Ia) and (Ib) and providing a higher AUC of compound (I) than compound (Ic). The Cmax and PK profile can be measured by orally administering compounds of the invention to one or more subjects, e.g. animals for example rats, minipigs, monkey or to a human being, wherein rat is a preferred subject, and hereafter measuring the concentration of compound (I) in plasma as described in Example 2 or Example 3. Thus, in one embodiment of the invention, a compound of formula (Id) has an observed PK profile avoiding the peak Cₘₐₓ observed for the known prodrugs (Ia) and (Ib) and providing a higher AUC of compound (I) than compound (Ic). Thus, in one embodiment of the invention, compounds of formula (Id) have a Cmax above about 90 pg/mL, such as in the range of about 90 pg/mL to about 600 pg/mL, such as in the range of about 90 pg/mL to about 400 pg/mL, such as in the range of about 100 pg/mL to 200 pg/mL, or such as in the range of about 200 pg/mL to about 300 pg/mL, or such as in the range of about 300 pg/mL to about 400 pg/mL. In another embodiment of the invention, the Cmax is below 3000 pg/mL, more preferably below 1000 pg/mL such as in the range of about 50 pg/mL to about 500 pg/mL.

In another embodiment of the invention, the compounds of formula (Id) have a AUC as measured over 24 hours above 1000 pg*h/mL, such as in the range of about 1500 to about 5000 pg*h/mL, such as in the range of about 1500 pg*h/mL to 4000 pg*h/mL, such as about 1600 pg*h/mL, or such as about 1700 pg*h/mL, or such as about 1800 pg*h/mL, or such as about 1900 pg*h/mL, or such as about 2000 pg*h/mL, or such as about 2100 pg*h/mL, or such as about 2200 pg*h/mL, or such as about 2300 pg*h/mL, or such as about 2400 pg*h/mL, or such as about 2500 pg*h/mL, or such as about 2600 pg*h/mL, or such as about 2700 pg*h/mL, or such as about 2800 pg*h/mL, or such as about 2900 pg*h/mL, or such as about 3000 pg*h/mL, or such as about 3100 pg*h/mL, or such as about 3200 pg*h/mL, or such as about 3300 pg*h/mL, or such as about 3400 pg*h/mL, or such as about 3500 pg*h/mL, or such as about 3600 pg*h/mL, or such as about 3700 pg*h/mL, or such as about 3800 pg*h/mL, or such as about 3900 pg*h/mL, or such as about 4000 pg*h/mL, or such as about 4100 pg*h/mL, or such as about 4200 pg*h/mL, or such as about 4300 pg*h/mL, or such as about 4400 pg*h/mL, or such as about 4500 pg*h/mL, or such as about 4600 pg*h/mL, or such as about 4700 pg*h/mL, or such as about 4800 pg*h/mL, or such as about 4900 pg*h/mL, or such as about 5000 pg*h/mL.

In an even more preferred embodiment of the invention, the compounds of formula (Id) have a Cmax above about 90 pg/mL, such as in the range of about 90 pg/mL to about 600 pg/mL, and an AUC as measured over 24 hours in the range of about 1500 to about 5000 pg*h/mL, such as in the range of about 1500 pg*h/mL to 4000 pg*h/mL.

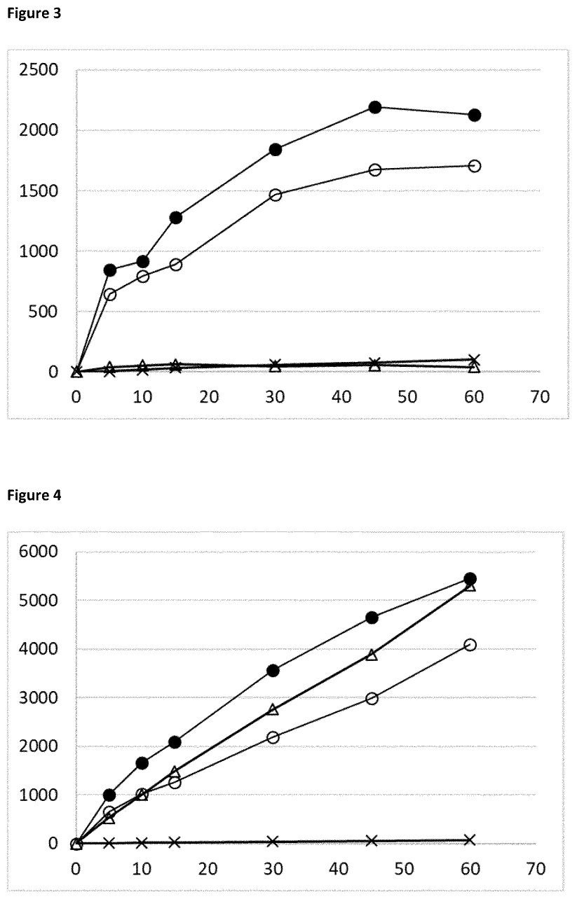

Example 4 demonstrates that compounds (II) and (III) are converted to compound (I) by incubation with human and rat liver S9 fractions (FIGS. 3 and 4). Further, compounds (II) and (III) of the invention were demonstrated to have increased conversion to compound (I) by incubation with human liver S9 as compared to prior art sulfate conjugate compounds (Id-iia) and (Id-iib) (FIG. 3).

Thus, in one embodiment of the invention, compounds of formula (Id) have increased conversion to compound (I) after incubation with human liver S9 as compared to prior art sulfate conjugate compounds (Id-iia) and (Id-iib). In a more specific embodiment of the invention, compounds of formula (Id) have increased conversion to compound (I) as compared to prior art sulfate conjugate compounds (Id-iia) and (Id-iib), when measured using the method as described in Example 4, or other equivalent methods known in the prior art.

Further, Example 3 demonstrates that the solubility at pH 6.0 of compounds of the invention, particularly compound (II) and compound (III), is higher than the solubility of the corresponding prior art compounds (Id-iia) and (Id-iib), see Table 3. Solubility of compounds of the invention can be measured by dissolving a compound into a solution, for example a solution comprising a buffer at a suitable acidic pH, such as for example a phosphate buffer at pH 6.0 such as by the method described in Example 3.

Thus, in one embodiment of the invention, compounds of formula (Id) have increased solubility at acidic pH compared to prior art sulfate conjugate compounds (Id-iia) and (Id-iib), such as having a measured solubility at pH 6.0 of above about 0.035 mg/mL, such as in the range of about 0.035 to about 0.01 mg/mL, or above about 0.1 mg/mL, such as in the range of about 0.1 mg/mL to about 1.0 mg/mL, such as about 0.2 mg/mL, or about 0.3 mg/mL, or about 0.4 mg/mL, or about 0.5 mg/mL, or about 0.6 mg/mL, or about 0.7 mg/mL, or about 0.8 mg/mL, or about 0.9 mg/mL, or about 1.0 mg/mL, or above about 1.0 mg/mL, such as in the range of about 1.0 mg/mL to about 5 mg/mL.

Accordingly, one aspect of the invention provides sulfamate derivatives of compound (I) or a pharmaceutically acceptable salt thereof.

Thus, in a first aspect, the invention provides a compound according to formula (Id) below

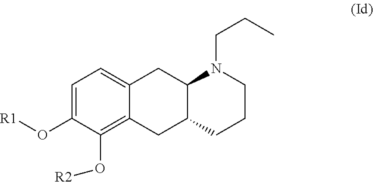

wherein R1 and R2 are each independently selected from H, and substituent (iii) below,

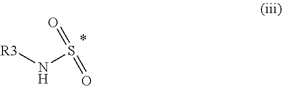

wherein * indicates the attachment point to oxygen,

wherein R3 is selected from H and COR4,

and wherein R4 is C1-C6 alkyl,

with the proviso that R1 and R2 cannot both be H,

or a pharmaceutically acceptable salt thereof.

Thus, in one embodiment of the invention, the invention provides a compound according to formula (Id) or a pharmaceutically acceptable salt thereof, wherein at least one of R1 or R2 is substituent (iii), such as the compound according to formula (Id) wherein R1 is substituent (iii), and R2 is H; or such as the compound according to formula (Id) wherein R1 is H and R2 is substituent (iii); or the compound according to formula (Id) wherein both R1 and R2 is substituent (iii).

In a preferred embodiment of the invention, a compound of the invention is a compound according to formula (Id) or a pharmaceutically acceptable salt thereof, wherein R1 is substituent (iii), and R2 is H; or a compound according to formula (Id) wherein R1 is H and R2 is substituent (iii).

In a preferred one embodiment of the invention, a compound according to formula (Id) or a pharmaceutically acceptable salt thereof is provided wherein R3 is H.

In another embodiment of the invention, a compound according to formula (Id) or a pharmaceutically acceptable salt thereof is provided wherein R3 is COR4, and wherein R4 is selected from H and C1-C6 alkyl. Such compounds include a compound according to formula (Id) or a pharmaceutically acceptable salt thereof wherein R3 is COR4, and wherein R4 is selected from the group consisting of hydrogen, methyl, ethyl, 1-propyl, 2-propyl, 1-butyl, 2-butyl, 2-methyl-2-propyl, 2-methyl-1-butyl, n-pentyl, isopentyl and n-hexyl. In an even more specific embodiment of the invention, a compound according to formula (Id) or a pharmaceutically acceptable salt thereof is provided wherein R3 is COR4, and wherein R4 is selected from the group consisting of methyl, ethyl, 1-propyl and 2-propyl.

In yet an even more specific embodiment of the invention, a compound according to formula (Id) or a pharmaceutically acceptable salt thereof is provided wherein R3 is COR4, and wherein R4 is H.

In yet an even more specific embodiment of the invention, a compound according to formula (Id) or a pharmaceutically acceptable salt thereof is provided wherein R3 is COR4, and wherein R4 is methyl.

In yet an even more specific embodiment of the invention, a compound according to formula (Id) or a pharmaceutically acceptable salt thereof is provided wherein R3 is COR4, and wherein R4 is ethyl.

In yet an even more specific embodiment of the invention, a compound according to formula (Id) or a pharmaceutically acceptable salt thereof is provided wherein R3 is COR4, and wherein R4 is 1-propyl.

In yet an even more specific embodiment of the invention, a compound according to formula (Id) or a pharmaceutically acceptable salt thereof is provided wherein R3 is COR4, and wherein R4 is 2-propyl.

In yet an even more specific embodiment of the invention, a compound according to formula (Id) or a pharmaceutically acceptable salt thereof is provided wherein R3 is COR4, and wherein R4 is 1-butyl.

In yet an even more specific embodiment of the invention, a compound according to formula (Id) or a pharmaceutically acceptable salt thereof is provided wherein R3 is COR4, and wherein R4 is 2-butyl.

In yet an even more specific embodiment of the invention, a compound according to formula (Id) or a pharmaceutically acceptable salt thereof is provided wherein R3 is COR4, and wherein R4 is 2-methyl-2-propyl.

In yet an even more specific embodiment of the invention, a compound according to formula (Id) or a pharmaceutically acceptable salt thereof is provided wherein R3 is COR4, and wherein R4 is 2-methyl-1-butyl.

In yet an even more specific embodiment of the invention, a compound according to formula (Id) or a pharmaceutically acceptable salt thereof is provided wherein R3 is COR4, and wherein R4 is n-pentyl.

In yet an even more specific embodiment of the invention, a compound according to formula (Id) or a pharmaceutically acceptable salt thereof is provided wherein R3 is COR4, and wherein R4 is isopentyl.

In yet an even more specific embodiment of the invention, a compound according to formula (Id) or a pharmaceutically acceptable salt thereof is provided wherein R3 is COR4, and wherein R4 is n-hexyl.

In a specific embodiment of the invention, a compound of the invention is a compound of formula (Id) or a pharmaceutically acceptable salt thereof, wherein at least one of R1 and R2 is substituent (iii), wherein R3 is H. Thus, in one embodiment of the invention, a compound of the invention is a compound of formula (Id) is selected from the group consisting of (4aR,10aR)-6-hydroxy-1-propyl-1,2,3,4,4a,5,10,10a-octahydrobenzo[g]quinolin-7-yl sulfamate of formula (II) below

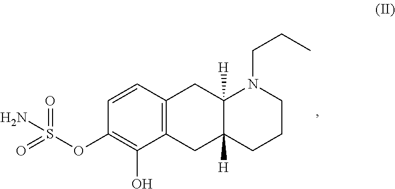 [SMILES: CCCN1CCC[C@@H]2Cc3c(ccc(OS(N)(=O)=O)c3O)C[C@H]21]

(4aR,10aR)-7-hydroxy-1-propyl-1,2,3,4,4a,5,10,10a-octahydrobenzo[g]quinolin-6-yl sulfamate of formula (III) below

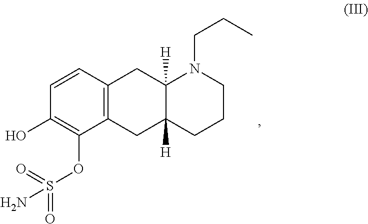 [SMILES: CCCN1CCC[C@@H]2Cc3c(ccc(O)c3OS(N)(=O)=O)C[C@H]21]

### (4aS,10aR)-4a-amino-1-propyl-1,2,3,4,4a,5,10,10a-octahydrobenzo[g]quinoline-6,7-diyl bis(sulfamate) of Formula (IV) Below

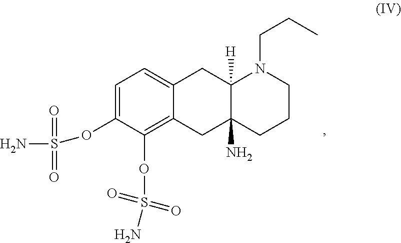 [SMILES: CCCN1CCC[C@]2(N)Cc3c(ccc(OS(N)(=O)=O)c3OS(N)(=O)=O)C[C@@H]12]

and a pharmaceutically acceptable salt of any one these compounds.

In a specific embodiment of the invention, a compound of the invention is a compound of formula (Id) or a pharmaceutically acceptable salt thereof, wherein at least one of R1 and R2 is substituent (iii), wherein R3 is H. Thus, in one embodiment of the invention, a compound of the invention is a compound of formula (Id) is selected from the group consisting of (4aR,10aR)-6-hydroxy-1-propyl-1,2,3,4,4a,5,10,10a-octahydrobenzo[g]quinolin-7-yl sulfamate of formula (II) below

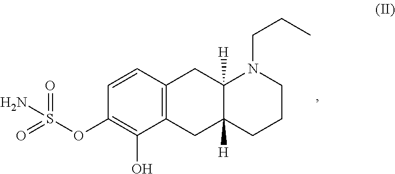 [SMILES: CCCN1CCC[C@@H]2Cc3c(ccc(OS(N)(=O)=O)c3O)C[C@H]21]

(4aR,10aR)-7-hydroxy-1-propyl-1,2,3,4,4a,5,10,10a-octahydrobenzo[g]quinolin-6-yl sulfamate of formula (III) below

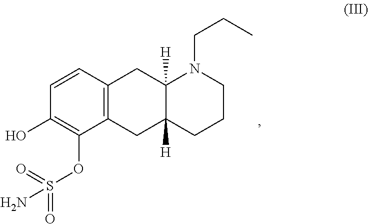 [SMILES: CCCN1CCC[C@@H]2Cc3c(ccc(O)c3OS(N)(=O)=O)C[C@H]21]

(4aR,10aR)-1-propyl-1,2,3,4,4a,5,10,10a-octahydrobenzo[g]quinoline-6,7-diyl bis(sulfamate) of formula (V) below

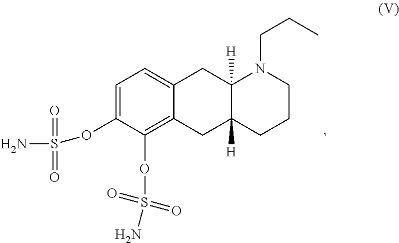 [SMILES: CCCN1CCC[C@@H]2Cc3c(ccc(OS(N)(=O)=O)c3OS(N)(=O)=O)C[C@H]21]

and a pharmaceutically acceptable salt of any one these compounds.

In a preferred embodiment of the invention, a compound of the invention is a compound of formula (Id) or a pharmaceutically acceptable salt thereof, wherein both R1 and R2 are substituent (iii), and wherein R3 is H. Accordingly, in a preferred embodiment of the invention, the compound of formula (Id) is (4aR,10aR)-1-propyl-1,2,3,4,4a,5,10,10a-octahydrobenzo[g]quinoline-6,7-diyl bis(sulfamate).

Example 2 demonstrates that the sulfamate conjugated compounds (II) and (III), which are examples of compounds according to formula (Id), wherein one of R1 and R2 are substituent (iii), and wherein R3 is H, are being converted to compound (I) in vivo.

Thus, in a preferred embodiment of the invention, a compound of the invention is a compound of formula (Id) or a pharmaceutically acceptable salt thereof, wherein one of R1 and R2 are H and the other is substituent (iii), wherein R3 is H.

Accordingly, in a preferred embodiment of the invention, a compound according to formula (Id), is selected from the group consisting of (4aR,10aR)-6-hydroxy-1-propyl-1,2,3,4,4a,5,10,10a-octahydrobenzo[g]quinolin-7-yl sulfamate of formula (II) below

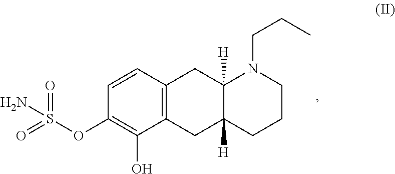 [SMILES: CCCN1CCC[C@@H]2Cc3c(ccc(OS(N)(=O)=O)c3O)C[C@H]21]

(4aR,10aR)-7-hydroxy-1-propyl-1,2,3,4,4a,5,10,10a-octahydrobenzo[g]quinolin-6-yl sulfamate of formula (III) below

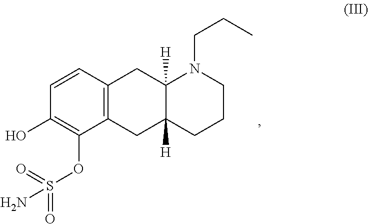 [SMILES: CCCN1CCC[C@@H]2Cc3c(ccc(O)c3OS(N)(=O)=O)C[C@H]21]

and a pharmaceutically acceptable salt of any of these compounds.

Thus, in yet a preferred embodiment of the invention a compound according to formula (Id) is (4aR,10aR)-6-hydroxy-1-propyl-1,2,3,4,4a,5,10,10a-octahydrobenzo[g]quinolin-7-yl sulfamate of formula (II) below

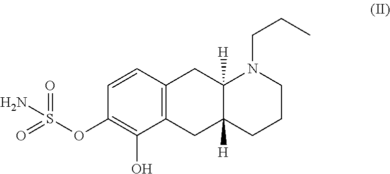 [SMILES: CCCN1CCC[C@@H]2Cc3c(ccc(OS(N)(=O)=O)c3O)C[C@H]21]

or a pharmaceutically acceptable salt thereof.

Thus, in another preferred embodiment of the invention a compound according to formula (Id) is (4aR,10aR)-7-hydroxy-1-propyl-1,2,3,4,4a,5,10,10a-octahydrobenzo[g]quinolin-6-yl sulfamate of formula (III) below

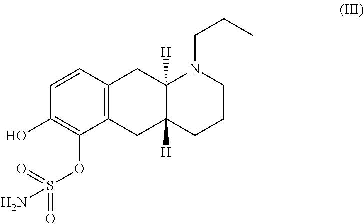 [SMILES: CCCN1CCC[C@@H]2Cc3c(ccc(O)c3OS(N)(=O)=O)C[C@H]21]

or a pharmaceutically acceptable salt thereof.

One embodiment of the invention provides a pharmaceutically acceptable salt of a compound according to formula (Id). In one embodiment, the pharmaceutically acceptable salt is an acid addition salt as defined herein above.

Determination of purity of compounds of the invention can be determined using different chromatographic methods optionally in combination with mass spectroscopy, e.g. LC/MS methods known in the art, e.g. as described in the example section.

In one embodiment of the invention, the compounds according to formula (Id) or a pharmaceutically acceptable salt thereof, is on an isolated form substantially free of the compound of formula (I).

In one embodiment of the invention, the compounds according to formula (Id) or a pharmaceutically acceptable salt thereof, is on an isolated form of above 90% purity, such as from about 90 to 99.99% purity, such as 95% purity, such as 96% purity, such as 97% purity, such as 98% purity, such as 99% purity, such as 99.5% purity.

Compounds of the invention can exist in different forms, such as amorphous or crystalline forms.

In one embodiment of the invention, the compounds according to formula (Id) or a pharmaceutically acceptable salt thereof, is in a solid form. In an even more preferred embodiment of the invention, a compound of formula (Id) or a pharmaceutically acceptable salt thereof in on a crystalline form.

Solid forms may also be amorphous solid forms. Thus, in one embodiment of the invention, a compound according to formula (Id) is on an amorphous solid form.

Included in this invention are also isotopically labelled compounds, which are similar to those claimed in formula (Id), wherein one or more atoms are represented by an atom of the same element having an atomic mass or mass number different from the atomic mass or mass number usually found in nature (e.g., 2H, 3H, 11C, 13C, 15N, and the like). Particular mention is made of 2H substituted compounds i.e. compounds wherein one or more H atoms are represented by deuterium.

In one embodiment of the invention one or more of the hydrogen atoms of the compound of formula (Id) are represented by deuterium. It is recognized that elements are present in natural isotopic abundances in most synthetic compounds and result in inherent incorporation of deuterium. However, the natural isotopic abundance of hydrogen isotopes such as deuterium is immaterial (about 0.015%) relative to the degree of stable isotopic substitution of compounds indicated herein.

Pharmaceutical Compositions

Another aspect of the invention provides a pharmaceutical composition comprising a therapeutically effective amount of a compound of formula (Id) or pharmaceutically acceptable salt thereof, and one or more pharmaceutically acceptable excipients.

In a preferred embodiment, a pharmaceutical composition comprises a compound of formula (Id) selected from the group consisting of compound (II), compound (III) and a pharmaceutically acceptable salt thereof.

A further aspect of the invention provides a process for making a pharmaceutical composition comprising a compound of formula (Id) or a pharmaceutically acceptable salt thereof, and more preferably the process is for making a pharmaceutical composition comprising a compound selected from the group consisting of compound (II) and compound (III) herein or a pharmaceutically acceptable salt thereof.

The pharmaceutical compositions according to the invention may be formulated with pharmaceutically acceptable excipients in accordance with conventional techniques such as those disclosed in Remington, “The Science and Practice of Pharmacy”, 22nd edition (2013), Edited by Allen, Loyd V., Jr.

The pharmaceutical composition comprising a compound of the present invention is preferably a pharmaceutical composition for oral administration. Pharmaceutical compositions for oral administration include solid oral dosage forms such as tablets, capsules, powders and granules; and liquid oral dosage forms such as solutions, emulsions, suspensions and syrups as well as powders and granules to be dissolved or suspended in an appropriate liquid.

Solid oral dosage forms may be presented as discrete units (e.g. tablets or hard or soft capsules), each containing a predetermined amount of the active ingredient, and preferably one or more suitable excipients. Where appropriate, the solid dosage forms may be prepared with coatings such as enteric coatings or they may be formulated so as to provide modified release of the active ingredient such as delayed or extended release according to methods well known in the art. Where appropriate, the solid dosage form may be a dosage form disintegrating in the saliva, such as for example an orodispersible tablet.

In a preferred embodiment of the invention, a pharmaceutical composition is for oral administration and selected from the group consisting of a tablet and a capsule.

Examples of excipients suitable for solid oral formulation include, but are not limited to, microcrystalline cellulose, corn starch, lactose, mannitol, povidone, croscarmellose sodium, sucrose, cyclodextrin, talcum, gelatin, pectin, magnesium stearate, stearic acid and lower alkyl ethers of cellulose. Similarly, the solid formulation may include excipients for delayed or extended release formulations known in the art, such as glyceryl monostearate or hypromellose. If solid material is used for oral administration, the formulation may for example be prepared by mixing the active ingredient with solid excipients and subsequently compressing the mixture in a conventional tableting machine; or the formulation may for example be placed in a hard capsule e.g. in powder, pellet or mini tablet form. The amount of solid excipient will vary widely but will typically range from about 25 mg to about 1 g per dosage unit.

Liquid oral dosage forms may be presented as for example elixirs, syrups, oral drops or a liquid filled capsule. Liquid oral dosage forms may also be presented as powders for a solution or suspension in an aqueous or non-aqueous liquid. Examples of excipients suitable for liquid oral formulation include, but are not limited to, ethanol, propylene glycol, glycerol, polyethylenglycols, poloxamers, sorbitol, poly-sorbate, mono and di-glycerides, cyclodextrins, coconut oil, palm oil, and water. Liquid oral dosage forms may for example be prepared by dissolving or suspending the active ingredient in an aqueous or non-aqueous liquid, or by incorporating the active ingredient into an oil-in-water or water-in-oil liquid emulsion.

Further excipients may be used in solid and liquid oral formulations, such as colourings, flavourings and preservatives etc.

Pharmaceutical compositions for parenteral administration include sterile aqueous and nonaqueous solutions, dispersions, suspensions or emulsions for injection or infusion, concentrates for injection or infusion as well as sterile powders to be reconstituted in sterile solutions or dispersions for injection or infusion prior to use. Examples of excipients suitable for parenteral formulation include, but are not limited to water, coconut oil, palm oil and solutions of cyclodextrins. Aqueous formulations should be suitably buffered if necessary and rendered isotonic with sufficient saline or glucose.

Other types of pharmaceutical compositions include suppositories, inhalants, creams, gels, dermal patches, implants and formulations for buccal or sublingual administration.

It is requisite that the excipients used for any pharmaceutical formulation comply with the intended route of administration and are compatible with the active ingredients.

### Use and Methods for Treatment

Other aspects of the invention provide a compound according to formula (Id) for use as a medicament, and methods of treatment wherein a compound according to formula (Id) is administered to a subject in need thereof.

Medical Indications and Conditions for Treatment

Compounds of the invention are conjugated versions of a dopamine agonist, and thus the compounds of the present invention are intended for use in treatment of neurodegenerative diseases and disorders such as Parkinson's disease and/or other conditions for which treatment with a dopamine agonist is therapeutically beneficial.

Therapeutic indications for which treatment with a dopamine agonist is therapeutically beneficial include a variety of central nervous system disorders characterized by motor and/or non-motor disturbances and for which part of the underlying pathophysiology is a dysfunction of the striatal-mediated circuitry. Such functional disturbances can be seen in neurodegenerative diseases, such as but not limited to Parkinson's disease (PD), Restless leg syndrome, Huntington's disease, and Alzheimer's disease and/or neuropsychiatric diseases such as, but not limited to schizophrenia, attention deficit hyperactivity disorder and drug addiction.

Thus, in one embodiment of the invention, a compound according to formula (Id) or a pharmaceutically acceptable salt thereof, or a pharmaceutical composition according to the invention, is for use in treatment of a neurodegenerative disease or disorder or a neuropsychiatric disease or disorder.

More specifically in one embodiment of the invention, the compound according to formula (Id) or a pharmaceutically acceptable salt thereof, or pharmaceutical compositions according to the invention, is for use in treatment of a neurodegenerative disease or disorder such as Parkinson's Disease, Huntington's disease, Restless leg syndrome or Alzheimer's disease; or a neuropsychiatric disease or disorder such as schizophrenia, attention deficit hyperactivity disorder or drug addiction.

The invention also provides a method for the treatment of a neurodegenerative disease or disorder such as Parkinson's Disease, Huntington's disease, Restless leg syndrome or Alzheimer's disease; or a neuropsychiatric disease or disorder such as schizophrenia, attention deficit hyperactivity disorder or drug addiction; which method comprises the administration of a therapeutically effective amount of a compound according to formula (Id) or pharmaceutically acceptable salt thereof, or a pharmaceutical composition according to the invention, to a patient in need thereof.

The compounds, being suitable for oral administration have the potential of providing a new treatment paradigm in Parkinson's Disease. Thus, in a preferred embodiment of the invention, the compounds of the invention according to formula (Id) or a pharmaceutically acceptable salt thereof, or pharmaceutical compositions according to the invention, are for use in a method for treatment of Parkinson's Disease.

In addition to neurodegenerative diseases and disorders, other conditions in which an increase in dopaminergic turnover may be beneficial are in the improvement of mental functions including various aspects of cognition. It may also have a positive effect in depressed patients, and it may also be used in the treatment of obesity as an anorectic agent and in the treatment of drug addiction. It may improve minimal brain dysfunction (MBD), narcolepsy, attention deficit hyperactivity disorder and potentially the negative, the positive as well as the cognitive symptoms of schizophrenia.

Restless leg syndrome (RLS) and periodic limb movement disorder (PLMD) are alternative indications, which are clinically treated with dopamine agonists. In addition, impotence, erectile dysfunction, SSRI induced sexual dysfunction, ovarian hyperstimulation syndrome (OHSS) and certain pituitary tumors (prolactinoma) are also likely to be improved by treatment with dopamine agonists. Dopamine is involved in regulation of the cardiovascular and renal systems, and accordingly, renal failure and hypertension can be considered alternative indications for the compounds of the invention.

Doses

The compounds of the present invention according to formula (Id) may be used for treatment in various doses needed for therapy. Thus, in one embodiment, the compound of the present invention is administered in an amount from about 0.0001 mg/kg body weight to about 5 mg/kg body weight per day. In particular, daily dosages may be in the range of 0.001 mg/kg body weight to about 2 mg/kg body weight per day. The exact dosages will depend upon the frequency and mode of administration, the sex, the age, the weight, and the general condition of the subject to be treated, the nature and the severity of the condition to be treated, any concomitant diseases to be treated, the desired effect of the treatment and other factors known to those skilled in the art.

A typical oral dosage for adults will be in the range of 0.01-100 mg/day of a compound of the present invention, such as 0.05-50 mg/day, such as 0.1-10 mg/day, or 0.1-5 mg/day, or 1 mg/day-10 mg/day, or 10 mg/day-15 mg/day, 15 mg/day-25 mg/day, 25 mg/day-35 mg/day, 35 mg/day-45 mg/day, or such as 55 mg/day-65 mg/day, or 70 mg/day-85 mg/day, or 80 mg/day-95 mg/day.

Conveniently, the compounds of the invention are administered in a unit dosage form containing said compounds in an amount of about 0.01 to 100 mg, such as of about 0.01 to 50 mg, such as 0.05 mg, 0.1 mg, 0.2 mg, 0.5 mg, 1 mg, 5 mg, 10 mg, 15 mg, 20 mg, 25 mg, 30 mg, 35 mg, 40 mg, 45 mg or up to 50 mg of a compound of the present invention.

Administration Routes

The pharmaceutical compositions comprising a compound of formula (Id), either as the sole active compound or in combination with another active compound, may be specifically formulated for administration by any suitable route such as the oral, rectal, nasal, buccal, sublingual, pulmonal, transdermal and parenteral (e.g. subcutaneous, intramuscular, and intravenous) route. In the context of the present invention the oral route is the preferred route of administration.

It will be appreciated that the route will depend on the general condition and age of the subject to be treated, the nature of the condition to be treated and the active ingredient.

A further aspect of the invention relates to a method for the treatment of a neurodegenerative disease or disorder such as Parkinson's Disease, Huntington's disease, Restless leg syndrome or Alzheimer's disease; or a neuropsychiatric disease or disorder such as schizophrenia, attention deficit hyperactivity disorder or drug addiction; which method comprises the administration of a therapeutically effective amount of a compound of formula (Id) or a pharmaceutically acceptable salt thereof, or pharmaceutical compositions according to the invention, to a patient in need thereof.

In a preferred embodiment of the invention, the method of treatment is for treatment of a neurodegenerative disease or disorder, and even more preferably for treatment of Parkinson's Disease.

Yet a further aspect of the invention relates to the use of a compound according to formula (Id) or a pharmaceutically acceptable salt thereof, or pharmaceutical compositions according to the invention, in the manufacture of a medicament for the treatment of a neurodegenerative disease or disorder such as Parkinson's Disease, Huntington's disease, Restless leg syndrome or Alzheimer's disease; or for the treatment of a neuropsychiatric disease or disorder such as schizophrenia, attention deficit hyperactivity disorder or drug addiction.

In a preferred embodiment of the invention, compounds of formula (Id) or a pharmaceutically acceptable salt thereof, or pharmaceutical compositions according to the invention, is used in the manufacture of a medicament for the treatment of a neurodegenerative disease or disorder, and even more preferably in the manufacture of a medicament for the treatment of Parkinson's Disease.

In one embodiment of the invention, the compounds of formula (Id), or a pharmaceutically acceptable salt thereof, or pharmaceutical compositions according to the invention, are for use as stand-alone treatment as the sole active compound.

In one embodiment of the invention, the compounds of formula (Id), or a pharmaceutically acceptable salt thereof, or pharmaceutical compositions according to the invention, are for use as stand-alone treatment of a neurodegenerative disease or disorder, such as Parkinson's Disease.

Combinations

In another embodiment of the invention, the compounds of formula (Id) or a pharmaceutically acceptable salt thereof, or pharmaceutical compositions according to the invention, may be used in combination with other agents useful in the treatment of a neurodegenerative disease or disorder such as Parkinson's disease.

In one embodiment of the invention, a compound selected from the group consisting of compounds (II), (III) and (IV) or a pharmaceutically acceptable salt of any one of these compounds is used in combination with other agents useful in the treatment of a neurodegenerative disease or disorder such as Parkinson's disease. In one embodiment of the invention, a compound selected from the group consisting of compounds (II), (III) and (V) or a pharmaceutically acceptable salt of any one of these compounds is used in combination with other agents useful in the treatment of a neurodegenerative disease or disorder such as Parkinson's disease. In an even more specific embodiment, a compound selected from the group consisting of compounds (II) and (III) or a pharmaceutically acceptable salt of any one of these compounds is used in combination with other agents useful in the treatment of a neurodegenerative disease or disorder such as Parkinson's disease.

The two compounds may be administered simultaneously or with a time gap between the administrations of the two compounds. The two compounds may be administered either as part of the same pharmaceutical formulation or composition, or in separate pharmaceutical formulations or compositions. The two compounds may be administered on the same day or on different days. They may be administered by the same route, such for example by oral administration, subcutaneous injection, by transdermal administration, by depot, by intramuscular injection or intravenous injection; or by different routes wherein one compound is for example administered orally or placed by depot and the other compound is for example injected. The two compounds may be administered by the same dosage regime or interval, such as once or twice daily, weekly, or monthly; or by different dosage regimes for example wherein one is administered once daily and the other is administered twice daily or weekly or monthly.

In some instances, the patient to be treated may already be in treatment with one or more other compounds useful in the treatment of a neurodegenerative disease or disorder when treatment with a compound of formula (Id), or a pharmaceutically acceptable salt thereof is initiated. In other instances, the patient may already be in treatment with a compound of formula (Id) when treatment with one or more other compounds useful in the treatment of a neurodegenerative disease or disorder is initiated. In other instances, the treatment with a compound of formula (Id) and treatment with one or more other compounds useful in the treatment of a neurodegenerative disease or disorder is initiated at the same time.

The compounds of the invention, or pharmaceutically acceptable salts thereof, or pharmaceutical compositions according to the invention, may be used in combination with one or more compounds typically used for treatment of a neurodegenerative disease or disorder such as Parkinson's disease. Thus, in one embodiment of the present invention, a compound of formula (Id), or a pharmaceutically acceptable salt thereof, or pharmaceutical compositions according to the invention, is used in combination with a compound selected from the group consisting of L-DOPA, droxidopa, foliglurax, MAO-B inhibitors such as selegiline or rasagiline, COMT inhibitors such as entacapone or tolcapone, adenosine 2a antagonists such as istradefylline, antiglutamatergic agents such as amantadine or memantine, acetylcholinesterase inhibitors such as rivastigmine, donepezil or galantamine and antipsychotic agents such as quetiapine, clozapine, risperidone, pimavanserin, olanzapine, haloperidol, aripiprazole and brexpiprazole.

In one embodiment of the invention a compound selected from the group consisting of compounds (II), (III) and (IV) or a pharmaceutically acceptable salt of any one of these compounds is used in combination with a compound selected from the group consisting of L-DOPA, droxidopa, foliglurax, MAO-B inhibitors such as selegiline or rasagiline, COMT inhibitors such as entacapone or tolcapone, adenosine 2a antagonists such as istradefylline, antiglutamatergic agents such as amantadine or memantine, acetylcholinesterase inhibitors such as rivastigmine, donepezil or galantamine and antipsychotic agents such as quetiapine, clozapine, risperidone, pimavanserin, olanzapine, haloperidol, aripiprazole and brexpiprazole.

In one embodiment of the invention a compound selected from the group consisting of compounds (II), (III) and (V) or a pharmaceutically acceptable salt of any one of these compounds is used in combination with a compound selected from the group consisting of L-DOPA, droxidopa, foliglurax, MAO-B inhibitors such as selegiline or rasagiline, COMT inhibitors such as entacapone or tolcapone, adenosine 2a antagonists such as istradefylline, antiglutamatergic agents such as amantadine or memantine, acetylcholinesterase inhibitors such as rivastigmine, donepezil or galantamine and antipsychotic agents such as quetiapine, clozapine, risperidone, pimavanserin, olanzapine, haloperidol, aripiprazole and brexpiprazole.

In an even more specific embodiment, a compound selected from the group consisting of compounds (II) and (III) or a pharmaceutically acceptable salt of any one of these compounds is used in combination with a compound selected from the group consisting of L-DOPA, droxidopa, foliglurax, MAO-B inhibitors such as selegiline or rasagiline, COMT inhibitors such as entacapone or tolcapone, adenosine 2a antagonists such as istradefylline, antiglutamatergic agents such as amantadine or memantine, acetylcholinesterase inhibitors such as rivastigmine, donepezil or galantamine and antipsychotic agents such as quetiapine, clozapine, risperidone, pimavanserin, olanzapine, haloperidol, aripiprazole and brexpiprazole.

In addition to small molecules, compounds for combination with compounds of the invention could also include emerging biologics approaches in treatments for neurodegenerative diseases or disorders such as for example antibodies targeting alpha-synuclein, Tau or A-beta proteins.

Thus, in one embodiment of the invention, the compounds of formula (Id) or a pharmaceutically acceptable salt thereof, or pharmaceutical compositions according to the invention, are to be used in combination with other agents for treatment of Parkinson's Disease such as a compound selected from the group consisting of L-DOPA, droxidopa, foliglurax, a MAO-B inhibitor such as selegiline or rasagiline, a COMT inhibitor such as entacapone or tolcapone, an adenosine 2a antagonist such as istradefylline, an antiglutamatergic agent such as amantadine or memantine, an acetylcholinesterase inhibitor such as rivastigmine, donepezil or galantamine, an antipsychotic agent such as quetiapine, clozapine, risperidone, pimavanserin, olanzapine, haloperidol, aripiprazole or brexpiprazole; or in combination with an antibody targeting alpha-synuclein, Tau or A-beta protein.

In one embodiment of the invention a compound selected from the group consisting of compounds (II), (III) and (IV) or a pharmaceutically acceptable salt of any one of these compounds is used in combination with a compound selected from consisting of L-DOPA, droxidopa, foliglurax, a MAO-B inhibitor such as selegiline or rasagiline, a COMT inhibitor such as entacapone or tolcapone, an adenosine 2a antagonist such as istradefylline, an antiglutamatergic agent such as amantadine or memantine, an acetylcholinesterase inhibitor such as rivastigmine, donepezil or galantamine, an antipsychotic agent such as quetiapine, clozapine, risperidone, pimavanserin, olanzapine, haloperidol, aripiprazole or brexpiprazole; or in combination with an antibody targeting alpha-synuclein, Tau or A-beta protein.

In one embodiment of the invention a compound selected from the group consisting of compounds (II), (III) and (V) or a pharmaceutically acceptable salt of any one of these compounds is used in combination with a compound selected from consisting of L-DOPA, droxidopa, foliglurax, a MAO-B inhibitor such as selegiline or rasagiline, a COMT inhibitor such as entacapone or tolcapone, an adenosine 2a antagonist such as istradefylline, an antiglutamatergic agent such as amantadine or memantine, an acetylcholinesterase inhibitor such as rivastigmine, donepezil or galantamine, an antipsychotic agent such as quetiapine, clozapine, risperidone, pimavanserin, olanzapine, haloperidol, aripiprazole or brexpiprazole; or in combination with an antibody targeting alpha-synuclein, Tau or A-beta protein.

In an even more specific embodiment, a compound selected from the group consisting of compounds (II) and (III) or a pharmaceutically acceptable salt of any one of these compounds is used in combination with a compound selected from consisting of L-DOPA, droxidopa, foliglurax, a MAO-B inhibitor such as selegiline or rasagiline, a COMT inhibitor such as entacapone or tolcapone, an adenosine 2a antagonist such as istradefylline, an antiglutamatergic agent such as amantadine or memantine, an acetylcholinesterase inhibitor such as rivastigmine, donepezil or galantamine, an antipsychotic agent such as quetiapine, clozapine, risperidone, pimavanserin, olanzapine, haloperidol, aripiprazole or brexpiprazole; or in combination with an antibody targeting alpha-synuclein, Tau or A-beta protein.

In one embodiment, compounds according to formula (Id), or a pharmaceutically acceptable salt thereof, or a pharmaceutical composition of the present invention is used as the sole medicament for treatment of a patient. In one embodiment, compounds according to formula (Id), or a pharmaceutically acceptable salt thereof, or a pharmaceutical composition of the present invention are used for treatment of patients who are not already in treatment with another drug selected from the list above.

One aspect of the invention provides the use of a compound of formula (Id) or pharmaceutically acceptable salt thereof, or a pharmaceutical composition according the invention in the manufacture of a medicament for the treatment of neurodegenerative disease or disorder such as Parkinson's Disease, Huntington's disease, Restless leg syndrome or Alzheimer's disease; or a neuropsychiatric disease or disorder such as schizophrenia, attention deficit hyperactivity disorder or drug addiction. In a preferred embodiment, the medicament is for the treatment of Parkinson's Disease.

### Exemplified Compounds of the Invention

TABLE 1
Overview of exemplified compounds of the invention

| Compound | | |
|---|---|---|
| no | Chemical name | Chemical structure |
| (II) | (4aR,10aR)-6-hydroxy-1-propyl- 1,2,3,4,4a,5,10,10a- octahydrobenzo[g]quinolin-7-yl sulfamate | 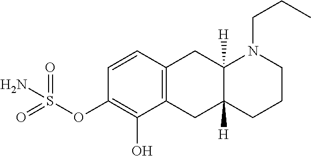 [SMILES: CCCN1CCC[C@@H]2Cc3c(ccc(OS(N)(=O)=O)c3O)C[C@H]21] |
| (III) | (4aR,10aR)-7-hydroxy-1-propyl- 1,2,3,4,4a,5,10,10a- octahydrobenzo[g]quinolin-6-yl sulfamate | 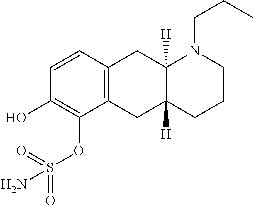 [SMILES: CCCN1CCC[C@@H]2Cc3c(ccc(O)c3OS(N)(=O)=O)C[C@H]21] |
| (IV) | (4aS,10aR)-4a-amino-1-propyl- 1,2,3,4,4a,5,10,10a- octahydrobenzo[g]quinoline-6,7-diyl bis(sulfamate) | 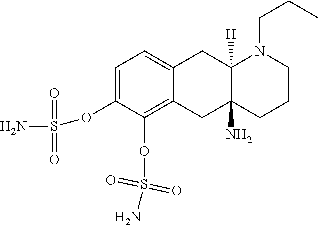 [SMILES: CCCN1CCC[C@]2(N)Cc3c(ccc(OS(N)(=O)=O)c3OS(N)(=O)=O)C[C@@H]12] |
| (V) | (4aR,10aR)-1-propyl-1,2,3,4,4a,5,10,10a- octahydrobenzo[g]quinoline-6,7-diyl bis(sulfamate) | 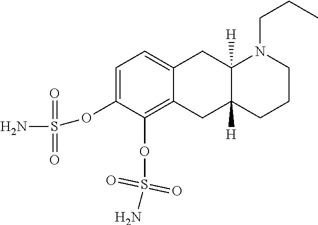 [SMILES: CCCN1CCC[C@@H]2Cc3c(ccc(OS(N)(=O)=O)c3OS(N)(=O)=O)C[C@H]21] |

### EXAMPLES

### Example 1—Preparation of the Compounds of the Invention

The compounds of the present invention of the general formula Id, wherein R1 and R2 are as defined above can be prepared by the methods outlined in the following examples. In the described methods, it is possible to make use of variants or modifications, which are themselves known to chemists skilled in the art or could be apparent to the person of ordinary skill in this art. Furthermore, other methods for preparing compounds of the invention will be readily apparent to the person skilled in the art in light of the following examples.

The starting materials used herein are available commercially or may be prepared by routine methods known in the art, such as those methods described in standard reference books such as “Compendium of Organic Synthetic Methods, Vol. I-XII” (published with Wiley-Interscience). Preferred methods include, but are not limited to, those described below.

More specifically the compound (4aR,10aR)-1-n-propyl-2H,3H,4H,4aH,5H,10H,10aH-benzo[g]quinoline-6,7-diol may be prepared according to the descriptions in e.g. WO 2009/026934.

The schemes are representative of methods useful in synthesizing the compounds of the present invention. They are not intended to constrain the scope of the invention in any way.

### Compound (II)

### (4aR,10aR)-6-hydroxy-1-propyl-1,2,3,4,4a,5,10,10a-octahydrobenzo[g]quinolin-7-yl sulfamate

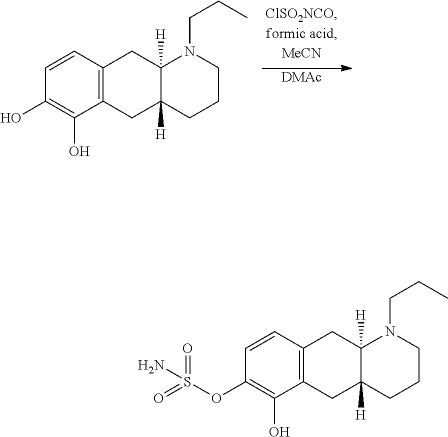

To a stirred solution of sulfurisocyanatidic chloride (4.07 g, 28.6 mmol) in acetonitrile (20 mL) was added formic acid (1.32 g, 28.7 mmol) dropwise at room temperature. The resulting mixture was stirred overnight at room temperature. To the above mixture was added (4aR,10aR)-1-n-propyl-2H,3H,4H,4aH,5H,10H,10aH-benzo[g]quinoline-6,7-diol (1.50 g, 5.7 mmol) and N,N-dimethylacetamide (10 mL). The resulting mixture was stirred for an additional 6 hours at room temperature. The reaction was quenched by the addition of ammonium hydroxide solution (10 mL) at room temperature. The resulting mixture was concentrated under reduced pressure.

The residue was purified by Prep-HPLC (Column: XBridge Shield RP18 OBD Column, 19*250 mm, 10 micrometer; Mobile Phase A: Water (10 mMol/L NH4HCO3), Mobile Phase B: ACN; Flow rate: 25 mL/min; Gradient: 17% B to 32% B in 10 minutes; 220 nm; Retention Time: 9.10 min) to yield (4aR,10aR)-6-hydroxy-1-propyl-2H,3H,4H,4aH,5H,10H,10aH-benzo[g]quinolin-7-yl sulfamate (406 mg).

LC-MS Instrument and protocol: Shimadzu LCMS-2020 fitted with a Shim-Pack XR-ODS C18, L=50 mm, D=3.0 mm column operated at 40° C. Mobile phase A: 0.05% TFA in water; mobile phase B: 0.05% TFA in acetonitrile. Flow rate 1.2 mL/min.

Gradient:

0-3.2 min A:B 95:5;

3.2 min-3.7 min: A:B 1:1;

3.7 min-4.75 min: A:B 0:1;

4.75 min-5.0 min: A:B 95:5.

LC-MS (MH+): m/z=341.2 Retention time (min)=1.523

1H NMR (400 MHz, DMSO) δ 7.01-6.99 (d, 1H), 6.62-6.60 (d, 1H), 2.94-2.911 (m, 1H), 2.85-2.79 (m, 1H), 2.72-2.66 (m, 1H), 2.51-2.49 (m, 1H), 2.45-2.28 (m, 1H), 2.16-2.06 (m, 3H), 1.85-1.82 (m, 1H), 1.64-1.40 (m, 5H), 1.09-1.06 (m, 1H), 0.86-0.83 (t, 3H).

### Compound (III)

### (4aR,10aR)-7-hydroxy-1-propyl-1,2,3,4,4a,5,10,10a-octahydrobenzo[g]quinolin-6-yl sulfamate

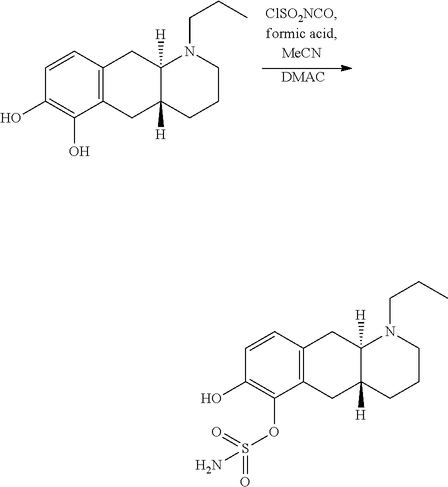

To a stirred solution of sulfurisocyanatidic chloride (4.07 g, 28.6 mmol) in acetonitrile (20 mL) was added formic acid (1.32 g, 28.7 mmol) dropwise at room temperature. The resulting mixture was stirred overnight at room temperature. To the above mixture was added (4aR,10aR)-1-n-propyl-2H,3H,4H,4aH,5H,10H,10aH-benzo[g]quinoline-6,7-diol (1.50 g, 5.7 mmol) and N,N-dimethylacetamide (10 mL). The resulting mixture was stirred for an additional 6 hours at room temperature. The reaction was quenched by the addition of ammonium hydroxide solution (10 mL) at room temperature. The resulting mixture was concentrated under reduced pressure. The residue was purified by Prep-HPLC (Column: XBridge Shield RP18 OBD Column, 19*250 mm, 10 micrometer; Mobile Phase A:Water (10 mMol/L NH₄HCO₃), Mobile Phase B: ACN; Flow rate: 25 mL/min; Gradient: 17% B to 32% B in 10 minutes; 254 nm; Retention time: 8.18 minutes) to yield (4aR,10aR)-7-hydroxy-1-propyl-2H,3H,4H,4aH,5H,10H,10aH-benzo[g]quinolin-6-yl sulfamate (297 mg).

LC-MS Instrument and protocol: Shimadzu LCMS-2020 fitted with a Shim-Pack XR-ODS C18, L=50 mm, D=3.0 mm column operated at 40° C. Mobile phase A: 0.05% TFA in water; mobile phase B: 0.05% TFA in acetonitrile. Flow rate 1.2 mL/min.

Gradient:

0-3.2 min A:B 95:5;

3.2 min-3.7 min: A:B 1:1;

3.7 min-4.75 min: A:B 0:1;

4.75 min-5.0 min: A:B 95:5.

LC-MS (MH+): m/z=341.2 Retention time (min)=1.42

¹H NMR (400 MHz, DMSO) δ 6.82-6.84 (d, 1H), 6.73-6.70 (d, 1H), 3.12-3.07 (m, 1H), 2.95-2.90 (m, 1H), 2.72-2.63 (m, 1H), 2.53-2.51 (m, 1H), 2.50-2.43 (m, 1H), 2.41-2.32 (m, 3H), 1.82-1.79 (m, 1H), 1.64-1.39 (m, 5H), 1.07-1.02 (m, 1H), 0.86-0.82 (t, 3H).

### Compound (V)

### (4aR,10aR)-1-propyl-1,2,3,4,4a,5,10,10a-octahydrobenzo[g]quinoline-6,7-diyl bis(sulfamate)

To a stirred solution of sulfurisocyanatidic chloride (4.07 g, 28.6 mmol) in acetonitrile (20 mL) was added formic acid (1.32 g, 28.7 mmol) dropwise at room temperature. The resulting mixture was stirred overnight at room temperature. To the above mixture was added (4aR,10aR)-1-propyl-2H,3H,4H,4aH,5H,10H,10aH-benzo[g]quinoline-6,7-diol (1.50 g, 5.7 mmol) and N,N-dimethylacetamide (10 mL). The resulting mixture was stirred for an additional 6 hours at room temperature. The reaction was quenched by the addition of ammonium hydroxide solution (10 mL) at room temperature. The resulting mixture was concentrated under reduced pressure. The residue was purified by Prep-HPLC (Column: XBridge Shield RP18 OBD Column, 19*250 mm, 10 micrometer; Mobile Phase A:Water (10 mMol/L NH₄HCO₃), Mobile Phase B: ACN; Flow rate: 25 mL/min; Gradient: 17% B to 32% B in 10 minutes; 254 nm) to yield (4aR,10aR)-1-propyl-1,2,3,4,4a,5,10,10a-octahydrobenzo[g]quinoline-6,7-diyl bis(sulfamate).

### Example 2—In Vivo Conversion to Dopamine Agonist (4aR,10aR)-1-Propyl-1,2,3,4,4a,5,10,10a-octahydro-benzo[g]quinoline-6,7-diol

The present example demonstrates that selected compounds of the invention are converted to compound (I) in vivo in rats.

PK Experiments in Rats

For the experiments relating to compounds (Ia), (Ib), (Ic), (Id-iia), (Id-iib), and (Id-iiab), the compounds were synthesized as described in WO2019101917, Liu et al., J. Med. Chem. (2006), 49: 1494-1498, Liu et al., Bioorganic Med. Chem. (2008) and WO 2009/026934. Blood samples of approximately 0.68 mL were drawn from the tail or sublingual vein and put into K₃EDTA tubes that had been pre-cooled and prepared with stabilizing aqueous solution consisting of 80 microL 100 mg/mL ascorbic acid and 40 microL inhibitor solution containing 0.5M citric acid and 100 mM D-saccharic acid 1,4 lactone in water. The tubes were inverted gently 6-8 times to ensure thorough mixing and then placed in wet ice. The collecting tube was placed in wet ice for up to 30 minutes until centrifugation (3000 G for 10 minutes at 4° C.). Once removed from the wet ice the centrifugation was initiated immediately. Immediately after end of centrifugation the samples were returned to wet ice. Two sub-samples of 130 microL plasma were transferred to each of two appropriately labelled cryo tubes containing 6.5 microL pre-cooled formic acid (20% in water v/v) The tube lid was immediately replaced, and the plasma solution was thoroughly mixed by inverting gently 6-8 times. The plasma samples were placed on water-ice as far as possible and stored frozen at nominally −70° C. within 60 minutes after sampling.

For experiments relating to compounds (II) and (III), the above protocol was used, with the exception that K₃EDTA tubes were prepared with stabilizing aqueous solution consisting of 80 microL 100 mg/mL ascorbic acid and 40 microL inhibitor solution containing 100 mg/mL citric acid, 21 mg/mL D-saccharic acid 1,4 lactone and 29 mg/mL tris-(2-carboxyethyl)phosphine (TCEP).

Plasma samples were analyzed by solid phase extraction or direct protein precipitation followed by UPLC-MS/MS. MS detection using electrospray in the positive ion mode with monitoring of specific mass-to-charge transitions for compound (I) using internal standards for correcting the response. The concentration-time data was analyzed, using standard software using appropriate noncompartmental techniques to obtain estimates of the derived PK parameters.

Instrumentation Used for Analysis of Compound (I) from Dosing Compound (Ia):

Mass spectrometer (LC-MS/MS) Waters Acquity-Sciex API 5000. Analytical column Waters BEH UPLC Phenyl 100×2.1 mm column, 1.7 micro meter particle size. Mobile phase A: 20 mM ammonium formate (aq)+0.5% formic acid. Mobile phase B: Acetonitrile. Gradient run from 95/5% to 2/98 in 6.1 minutes. Flow rate 0.5 mL/min. MRM monitoring (multiple reaction monitoring) of test item and the added analytical standards.

Dosing and Blood Sampling Compound (Ia):

Han Wistar rats were supplied by Charles River Laboratories, Sulzfeld, Germany. An artificial, automatically controlled, light and dark cycle of 12 hours was maintained. The rats received a standard laboratory diet from Brogaarden (Altromin 1324 pellets). The rats had unrestricted access to the diet. During the study (a 4-week toxicity study) the rats received once daily doses of (Ia) orally by gavage. From rats given 300 micrograms/kg (Ia), blood samples) from 3 male satellite animals were collected on the following time points at Day 29: 0.5, 1, 2, 4, 6, 8, 12 and 24 hours after dosing.

Instrumentation Used for Analysis of Compound (I) from Dosing of Compound (Ib):

Mass spectrometer (LC-MS/MS) Waters Acquity-Sciex API 5000. Analytical column Waters BEH UPLC Phenyl 100×2.1 mm column, 1.7 micrometer particle size. Mobile phase A: 20 mM ammonium formate (aq)+0.5% formic acid. Mobile phase B: Acetonitrile. Gradient run from 95/5% to 2/98 in 6.1 minutes. Flow rate 0.5 mL/min. MRM monitoring of test item and the added analytical standards.

Dosing and Blood Sampling Compound (Ib):

Han Wistar rats were supplied by Charles River Laboratories, UK. An artificial, automatically controlled, light and dark cycle of 12 hours was maintained. The rats received a standard laboratory diet (Teklad 2014C Diet). The rats had unrestricted access to the diet. During the study (a 26-week toxicity study) the rats received once daily doses of (Ib) orally by gavage. From rats given 300 micrograms/kg (Ib), blood samples from 3 male satellite animals were collected on the following time points at day 182: 0.5, 1, 2, 4, 8 and 24 hours after dosing.

### Instrumentation Used for Analysis of Compound (I) from Dosing of Prior Art Compounds (Ic), and (4aR,10aR)-7-hydroxy-1-propyl-1,2,3,4,4a,5,10,10a-octahydrobenzo[g]quinolin-6-yl hydrogen sulfate (compound Id-iia), and (4aR,10aR)-6-hydroxy-1-propyl-1,2,3,4,4a,5,10,10a-octahydrobenzo[g]quinolin-7-yl hydrogen sulfate (compound Id-iib), (4aR,10aR)-1-propyl-1,2,3,4,4a,5,10,10a-octahydrobenzo[g]quinoline-6,7-diyl bis(hydrogen sulfate) (Compound (Id-iiab))

Mass spectrometer (LC-MS/MS) Waters Acquity-Waters Xevo TQ-S. Analytical column Acquity BEH C18 100×2.1 mm, 1.7 micrometer. Mobile phase A: 20 mM NH₄—Formate+0.2% formic acid. Mobile phase B: Acetonitrile+0.2% formic acid. Gradient run from 95/5% to 5/95% in 11.0 min. Flow rate 0.3 mL/min. MRM monitoring of test item and the added analytical standards.

Dosing and Blood Sampling for Compounds (Id-iia) and (Id-iib):

Han Wistar rats were supplied by Charles River Laboratories, Wiga GmbH, Germany. An artificial, automatically controlled, light and dark cycle of 12 hours was maintained. The rats received a standard laboratory diet from Brogaarden (Altromin 1324 pellets). The rats had unrestricted access to the diet. Male Han Wistar rats were dosed a single oral gavage administration of (Id-iia) and (Id-iib) respectively, orally by gavage. Rats were given 392 microgram/kg (Id-iia) and (Id-iib)), blood samples from 3 male animals were collected on the following time points at Day 1: 1, 2, 4, 6, 8, and 24 hours after dosing.

Dosing and Blood Sampling for Compounds (Ic) and (Id-iiab):

Han Wistar rats were supplied by Envigo, UK. An artificial, automatically controlled, light and dark cycle of 12 hours was maintained. The rats received a standard laboratory diet Teklad 2014C. The rats had unrestricted access to the diet. Male Han Wistar rats were dosed a single oral gavage administration of (Ic) and (Id-iiab), respectively, orally by gavage. Rats were given 703 microgram/kg (Id-iiab) and 494 microgram/kg (Ic). Blood samples from 3 male animals were collected on the following time points at Day 1: 1, 2, 4, 6, 8, and 24 hours after dosing.

Dosing and Blood Sampling for Compounds (II) and (III):

Han Wistar rats were supplied by Envigo, UK. An artificial, automatically controlled, light and dark cycle of 12 hours was maintained. The rats received a standard laboratory diet Teklad 2014C. The rats had unrestricted access to the diet. Male Han Wistar rats were dosed a single oral gavage administration of compound (II) or (III). Rats were given 390 microgram/kg of compound (II) or 390 microgram/kg of compound (III). Blood samples from 3 male animals were collected on the following time points at Day 1: 5 mins, 0.25 hours, 0.5 hours, 1 hours, 2 hours, 4 hours, 8 hours, and 24 hours after dosing.

Instrumentation Used for Analysis of Compound (I) from Dosing of Compounds (II) and (III):

Mass spectrometer (LC-MS/MS) Waters Acquity-Waters Xevo TQ-S. Analytical column Acquity BEH C18 100×2.1 mm, 1.7 micrometer. Mobile phase A: 20 mM NH₄—Formate+0.2% formic acid. Mobile phase B: Acetonitrile+0.2% formic acid. Gradient run from 95/5% to 5/95% in 9.1 minutes. Flow rate 0.3 mL/min. MRM monitoring of test item and the added analytical standards.

The measured mean plasma concentrations from 3 subjects per timepoint of (4aR,10aR)-1-Propyl-1,2,3,4,4a,5,10,10a-octahydro-benzo[g]quinoline-6,7-diol (compound (I)) in plasma at various time points after dosing compound (II) and (III) is shown in FIGS. 2 and 1, respectively.

Table 2 below further summarizes PK parameters based on the measured mean plasma concentrations.

The results in Table 2 indicate that the plasma exposure throughout 24 hours for the tested compounds (II) and (III) is lower than the corresponding exposure observed for prior art compounds (Ia), (Ib), while higher than the corresponding exposure for prior art compound (Ic), which was found not to be useful as a prodrug. Further, the Cmax of compound (I) observed after dosing both compounds (II) and (III) are lower than what is achievable from dosing prior art compounds (Ia) and (Ib), and the prior art sulfate conjugate compounds (Id-iia), and (Id-iib).

Since the peak concentrations (Cmax) of compound (I) which are expected to drive the side effects are lower for compounds (II) and (III), higher doses might be administered of the compounds of the invention to potentially achieve higher overall plasma concentrations of compound (I) compared to what is achievable from dosing compounds (Ia), (Ib), (Id-iia), and (Id-iib).

For compound (II), Tmax is observed after 1 hour, similar to prior art compounds (Ia), (Ib), and (Ic), whereas the prior art compounds (d-iia), (Id-iib) and (Id-iiab) had Tmax some hours later.

TABLE 2
PK parameters for (4aR,10aR)-1-Propyl-1,2,3,4,4a,5,10,10a-
octahydro-benzo[g]quinoline-6,7-diol (compound (I))
after oral dosing of 0.300 mg/kg (Ia), 0.300 mg/kg
(Ib), 0.494 mg/kg (Ic), 0.392 mg/kg of (Id-iia), 0.392 mg/kg
(Id-iib), 0.703 mg/kg (Id-iiab), 0.39 mg/kg compound (II) and
0.39 mg/kg compound (III) to Wistar rats according to Example 2

| | | | | | Expo- |
|---|---|---|---|---|---|
| | | | | | sure_at |
| | Com- | Tₘₐₓ | Cₘₐₓ | AUC₀₋₂₄ | 24 h |
| | pound | (h) | (pg/mL) | (pg * h/mL) | (pg/mL) |
| Prodrugs | (Ia) | 1.0 | 3160 | 13600 | 48 ± 26 |
| in the | (Ib) | 1.0 | 4990 | 31000 | 147 ± 28 |
| state of | (Ic) | 1.0 | 14 | 104 | N/A |
| the art | (Id-iia) | 6.0 | 945 | 11300 | 192 ± 14 |
| | (Id-iib) | 8.0 | 665 | 7800 | 166 ± 94 |
| | (Id-iiab) | 24 | 68 | 1040 | 68 ± 38 |
| Com- | (II) | 1 | 328 | 3370 | 177 |
| pounds | (III) | 24 | 109 | 1820 | 109 |
| of the | | | | | |
| invention | | | | | |

### Example 3—Solubility of Compounds of the Invention

The following example demonstrates that compounds of the invention have a higher solubility at pH 6.0 compared to sulfate derivative compounds of the prior art.

### Experimental Procedure

For compound (III), a sample was prepared with excess compound (III) to establish a saturated solution. 4.2 mg of compound (III) was transferred to a vial and 0.5 mL buffer (25 mM phosphate buffer, NaH₂PO₄/Na₂HPO₄, pH 6) was added. The sample was mixed by rotation for 24 hours. pH of the sample was measured at t=0 (prior to rotation) and at t=24 h (after 24 h of rotation). Subsequently, some of the sample was filtered with 0.22 micrometer polyvinylidene difluoride filter, first few drops discarded, and content of dissolved compound (III) was quantified by reverse phased HPLC (C₁₈, 3.5 micrometer, 4.6×150 mm column) with eluent 20 mM ammonium buffer pH 2.4 with methanol, 80:20.

The remaining undissolved material was extracted, and air dried for approximately 20 minutes on a metal plate prior to analysis by X-ray powder diffractogram (XRPD). XRPD diffractograms were obtained on a PANalytical X'Pert PRO X-Ray Diffractometer using CuKa1 radiation (λ=1.5406 Å). The samples were measured in reflection mode in the 2θ-range 3-39.9° using an X'celerator detector.

For compound (Id-iib) a similar analysis was performed using 0.799 mg of compound (Id-iib) in 0.5 mL buffer.

In addition, physical and chemical property values were predicted with ADMET Predictor™ version 10.3.0.7, Simulations Plus, Inc.

TABLE 3
demonstrates predicted Log P and Log D
values and measured solubility for the compounds:

| | | | | | Measured |
|---|---|---|---|---|---|
| | | | pKa | pKa | solubility |
| Compound name | Log P | Log D | (base) | (acid) | at pH 6 |
| Compound (Id-iia) | 0.88 | 0.87 | 9.17 | −0.45 | — |
| Compound (Id-iib) | 0.88 | 0.87 | 9.2 | −0.46 | 0.037 mg/mL |
| Compound (II) | 2.09 | 1.11 | 9.32 | 8.33 | — |
| Compound (III) | 2.03 | 1.24 | 9.23 | 8.12 | 0.89 mg/mL |

Results:

The measured solubility of compound III was determined to 0.89 mg/mL. The crystalline form prior to and after mixing with buffer was identical indicating no solid form change.

For the prior art sulfate derivate compound (Id-iib) the solubility was determined to 0.037 mg/mL. The crystalline form prior to and after mixing with buffer was identical indicating no solid form change.

The sulfate derivative compounds (Id-iib) and (Id-iia) are structurally very similar, resulting in very similar predicted pKa values and Log P and D. Therefore, the solubility of compound (Id-iia) is expected to be very similar to the measured solubility of compound (Id-iib).

Further, sulfamate derivative compounds (II) and (III) are structurally very similar, resulting in very similar predicted pKa values and Log P and D. Therefore, the solubility of compound II is expected to be very similar to the measured solubility of compound Ill.

To summarize, the data of the present example demonstrated that compounds of the invention have higher solubility than sulfate derivatives of the prior art.

### Example 4: Conversion of the Compounds of the Invention in Rat and Human Liver S9

The compounds (II), (III), (Id-iia), and (Id-iib) were incubated separately at 1 μM with rat and human liver S9 fraction suspended in 50 mM phosphate buffer at pH 7.4 with 5 mM MgCl₂, 200 U/mL superoxide dismutase and 1 mM ascorbic acid. The human liver S9 (pool from 50 donors) and the rat liver S9 (from Wistar Han rats, pool of 240) fractions were purchased from SekisuiXenoTech, USA. The protein concentration in the incubation was 4 mg/mL. The incubations were performed in 96-well plates at 37° C. using an automated Hamilton liquid handling system with a total incubation volume of 150 microliter per well. The compounds were each incubated in triplicates in three wells per time point. The liver S9 suspensions were pre-incubated at 37° C. and the reactions started by addition of stock solutions of the test compounds to the incubation wells. The test compounds were dissolved in dimethyl sulfoxide (DMSO) and further diluted in water before spiking the stock solution into the incubation wells. The final DMSO concentration in the incubations was 0.1%. The test compounds were incubated with liver S9 fractions for 0, 5, 10, 15, 30, 45 and 60 minutes. The incubations were terminated by transferring 100 microliter of the incubation samples to a 96-well plate containing 100 microliter of cold (approximately +4° C.) stop reagent containing 2% formic acid, 20 mg/mL ascorbic acid, 10 mg/mL citric acid and 3 mg/mL TCEP (tris(2-carboxyethyl)phosphine). After mixing, the samples were transferred to Nunc 1.0 mL cryo tubes and immediately frozen at −80° C. until analysis. Concentrations of compound (I) were determined after solid-phase extraction of the samples followed by LC-MS/MS analysis. The increase in concentration of compound (I) at the different time points was calculated by subtracting the concentration of compound (I) in the sample taken at time 0 from the concentrations of compound (I) in samples taken at the different time points.

Instrumentation Used for Analysis of Compound (I) from Incubation of Compounds (II), (III), (Id-iia) and (Id-iib):

Mass spectrometer (LC-MS/MS) Waters Acquity-Waters Xevo TQ-S. Analytical column Acquity BEH C18 50×2.1 mm, 1.7 micrometer. Mobile phase A: 0.2% formic acid in 20 mM NH4-Formate. Mobile phase B: 0.2% formic acid in acetonitrile. Gradient run from 5% B to 95% B in 3.5 minutes. Flow rate 0.6 mL/min. Multiple reaction monitoring (MRM) with positive polarization was applied to monitor precursor-product ion pair transitions of compound (I) and its deuterated internal standard.

Results:

FIGS. 3 and 4 show the measured increase in concentration of compound (I) at different time points after incubation of S9 fractions with compounds (II), (III), (Id-iia) and (Id-iib) for both human liver S9 (FIG. 3) and rat liver S9 (FIG. 4). The data based on the human S9 liver fraction (FIG. 3) show that the compounds (II) and (III) of the invention have increased conversion to compound (I) in human liver S9 fraction, compared to the prior art sulfate conjugates (compound (Id-iia) and compound (Id-iib). For the rat liver S9 incubation it is seen that compound (II), compound (III), and compound (Id-iib) is converted to compound (I).

### REFERENCES

U.S. Pat. No. 4,543,256
WO2001/078713
WO 02/100377
WO2009/026934
WO2009/026935
WO2010/097092
WO2019101917
Alexander et Crutcher, (1990) Trends in Neuroscience 13: 266-71;
Bibbiani et al., Chase Experimental Neurology (2005), 192: 73-78;
Campbell et al., Neuropharmacology (1982); 21(10): 953-961;
Cannon et al., J. Heterocyclic Chem. (1980); 17: 1633-1636;
Cavero and Guillon, J. Pharmacol. Toxicol. Methods (2014), 69: 150-161;
Delong, (1990) Trends in Neuroscience 13: 281-5; Elger W, Schwarz S, Hedden A et al.: Sulfamates of various estrogens are prodrugs with increased systemic and reduced hepatic estrogenicity at oral application. J. Steroid. Biochem. Mol. Biol. (1995) 55:395-403
Elger, W; Barth, A; Hedden, A; Reddersen, G; Ritter, P; Schneider, B; Züchner, J; Krahl, E; Müller, K; Oettel, M; Schwarz, S; “Estrogen sulfamates: a new approach to oral estrogen therapy”; Reproduction, fertility and development (2001) 13 (4) p. 297-305
Gerfen et al, Science (1990) 250: 1429-32;
Giardina and Williams; CNS Drug Reviews (2001), Vol. 7 (3): 305-316;
Grosset et al., Acta Neurol Scand. (2013), 128:166-171;
Hauser et al., Movement Disorders (2016), Vol. 32 (9): 1367-1372;
Liu et al., J. Med. Chem. (2006), 49: 1494-1498;
Liu et al., Bioorganic Med. Chem. (2008), 16: 3438-3444;
Poewe et al., Nature Review, (2017) vol 3 article 17013: 1-21;
Sprenger and Poewe, CNS Drugs (2013), 27: 259-272;
Sozio et al., Exp. Opin. Drug Disc. (2012); 7(5): 385-406;
Stahl and Wermuth (Eds) “Handbook of Pharmaceutical salts. Properties, selection, and use”, Wiley-VCH, 2008
Stain-Texier et al., Drug Metab. and Disposition (1998) 26 (5): 383-387;
Wiley-Interscience (publisher): Compendium of Organic Synthetic Methods, Vol. I-XII

1. A compound according to formula (Id) below 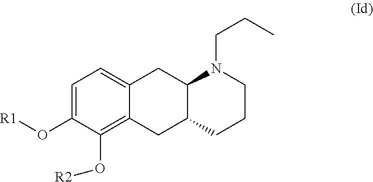
wherein R1 and R2 are each independently selected from H, and substituent (iii) below, 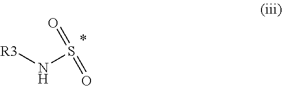
wherein * indicates the attachment point to oxygen,
wherein R3 is selected from H and COR4,
and wherein R4 is selected from H and C1-C6 alkyl,
with the proviso that R1 and R2 cannot both be H,
or a pharmaceutically acceptable salt thereof.

2. The compound according to claim 1 or a pharmaceutically acceptable salt thereof, wherein one of R1 or R2 is substituent (iii) and the other one is H.

3. The compound according to claim 1 or a pharmaceutically acceptable salt thereof, wherein both R1 and R2 are substituent (iii).

4. The compound according to claim 1, or a pharmaceutically acceptable salt thereof, wherein R3 is H.

5. The compound according to claim 1 or a pharmaceutically acceptable salt thereof, wherein the compound is selected from the group consisting of (4aR,10aR)-6-hydroxy-1-propyl-1,2,3,4,4a,5,10,10a-octahydrobenzo[g]quinolin-7-yl sulfamate of formula (II), and (4aR,10aR)-7-hydroxy-1-propyl-1,2,3,4,4a,5,10,10a-octahydrobenzo[g]quinolin-6-yl sulfamate of formula (III) below 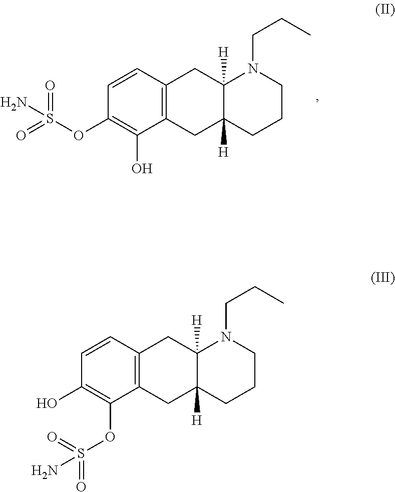 [SMILES: CCCN1CCC[C@@H]2Cc3c(ccc(OS(N)(=O)=O)c3O)C[C@H]21 ; CCCN1CCC[C@@H]2Cc3c(ccc(O)c3OS(N)(=O)=O)C[C@H]21]
and a pharmaceutically acceptable salt thereof.

6. The compound according to claim 1 or a pharmaceutically acceptable salt thereof, wherein said compound is on an isolated form substantially free of the compound of formula (I)

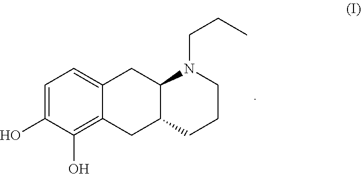 [SMILES: CCCN1CCC[C@@H]2Cc3c(ccc(O)c3O)C[C@H]21]

7. The compound or pharmaceutically acceptable salt thereof according to claim 1, wherein said compound or pharmaceutically acceptable salt thereof is in a solid form.

8. A pharmaceutically acceptable salt of the compound according to claim 1.

9. (canceled)

10. A pharmaceutical composition comprising a therapeutically effective amount of a compound or pharmaceutically acceptable salt thereof according claim 1, and one or more pharmaceutically acceptable excipients.

11. The pharmaceutical composition according to claim 10, wherein said pharmaceutical composition is an oral pharmaceutical composition such as a tablet or a capsule for oral administration.

12.-15. (canceled)

16. A method for the treatment of a neurodegenerative disease or disorder such as Parkinson's Disease, Huntington's disease, Restless leg syndrome or Alzheimer's disease; or a neuropsychiatric disease or disorder such as schizophrenia, attention deficit hyperactivity disorder or drug addiction; which method comprises the administration of a therapeutically effective amount of a compound or pharmaceutically acceptable salt thereof according to claim 1, to a patient in need thereof.

17. The method according to claim 16, for treatment of Parkinson's Disease.

18. The method of claim 16, comprising the administration of a therapeutically effective amount of a compound or pharmaceutically acceptable salt thereof according to claim 5.

19. The method of claim 17, comprising the administration of a therapeutically effective amount of a compound or pharmaceutically acceptable salt thereof according to claim 5.

20. The pharmaceutical composition of claim 10, comprising a therapeutically effective amount of a compound or pharmaceutically acceptable salt thereof according claim 5.

21. A pharmaceutically acceptable salt of the compound according to claim 5.

22. The compound according to claim 2 or a pharmaceutically acceptable salt thereof, wherein both R1 and R2 are substituent (iii).

23. The compound according to claim 2, or a pharmaceutically acceptable salt thereof, wherein R3 is H.

24. The compound according to claim 3, or a pharmaceutically acceptable salt thereof, wherein R3 is H.

25. The compound according to claim 5, or a pharmaceutically acceptable salt thereof, wherein said compound is on an isolated form substantially free of the compound of formula (I) 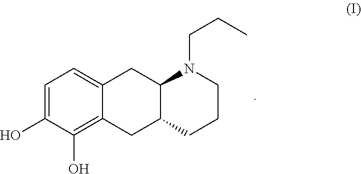 [SMILES: CCCN1CCC[C@@H]2Cc3c(ccc(O)c3O)C[C@H]21]
